(19) United States
(12) Patent Application Publication
(10) Pub. No.: US 2014/0201858 A1
(43) Pub. Date: Jul. 17, 2014

(54) METHODS FOR SITE-SPECIFIC GENETIC MODIFICATION IN STEM CELLS USING XANTHOMONAS TAL NUCLEASES (XTN) FOR THE CREATION OF MODEL ORGANISMS

(71) Applicants: Eric M. Ostertag, Lexington, KY (US); John S. Crawford, Lexington, KY (US); J. Keith Joung, Winchester, MA (US)

(72) Inventors: Eric M. Ostertag, Lexington, KY (US); John S. Crawford, Lexington, KY (US); J. Keith Joung, Winchester, MA (US)

(73) Assignee: Transposagen Biopharmaceuticals, Inc, Lexington, KY (US)

(21) Appl. No.: 14/118,544

(22) PCT Filed: May 17, 2012
(86) PCT No.: PCT/US12/38465

§ 371 Date: Jan. 27, 2014

Related U.S. Application Data
(60) Provisional application No. 61/487,267, filed on May 17, 2011.

(51) Int. Cl. C12N 15/85

(52) U.S. Cl. CPC C12N 15/8509
USPC 800/17; 435/325; 435/346; 435/419; 800/8; 800/14; 800/18; 800/19; 800/20; 800/22; 800/298; 800/312; 800/317.2; 800/317.3; 800/317.4; 800/320.1; 800/320.2; 800/320.3

(57) ABSTRACT

The invention relates to organisms and compositions comprising one or more stem cells or one or more embryos, wherein the one or more stem cells or one or more embryos comprise one or more of the following mutations: (i) a deletion mutation; (ii) a knockout mutation; and/or (iii) an addition of a heterologous nucleic acid sequence; wherein the one or more mutations of (i), (ii), and/or (iii) are site-specific mutations caused by a Xanthomonas TAL nuclease (XTN). The invention also relates to method of mutating an embryo, iPS cell, stem cell, or more particularly a spermatogonial stem cell by exposing the nucleic acid sequence contained within such embryos or cell with a Xanthomonas TAL nuclease.

### BACKGROUND OF THE INVENTION

Genetic modification is a process whereby an existing DNA sequence is altered or a new genetic sequence is added in a cell's or organism's genome. Site-specific genetic modification is the intentional alteration of a specific DNA sequence of a cell or organism. Oftentimes, a DNA sequence comprising a gene or gene fragment is chosen. This alteration of the targeted gene may result in a change in the level of RNA and/or protein that is encoded by that gene, or the alteration may result in the targeted gene encoding a different RNA or protein than the untargeted gene. The modified genome may be studied in the context of a cell, or, more preferably, in the context of a genetically modified organism.

Genetically modified organisms are among the most useful research tools in the biological sciences, as well as having agricultural, pharmaceutical and biotechnology applications. An example of a genetically modified organism is a knockout organism which harbors a genetic modification that results a loss of function to a gene and its encoded protein. Another example of a genetically modified organism is a knockin organism which contains an endogenous gene replaced with a heterologous (i.e., foreign) gene, or gene fragment. Genetically modified organisms will pass their genetic changes to their progeny if the changes have been incorporated into the organism's germ cells (i.e. sperm or oocytes).

Genetically modified organisms exhibiting clinically relevant phenotypes are valuable for drug discovery and development and for drug target identification. For example, mutation of somatic or germ cells facilitates the production of genetically modified offspring or cloned organisms having a phenotype of interest. Such organisms have a number of uses, for example as models of physiological disorders (e.g., of human genetic diseases) that are useful for screening the efficacy of candidate therapeutic compounds or compositions for treating or preventing such physiological disorders. Furthermore, identifying the gene(s) responsible for the phenotype provides potential drug targets for modulating the phenotype and, when the phenotype is clinically relevant, for therapeutic intervention. In addition, the manipulation of the genetic makeup of organisms and the identification of new genes have important uses in agriculture, for example in the development of new strains of animals and plants having higher nutritional value or increased resistance to environmental stresses (such as heat, drought, or pests) relative to their wild-type or non-mutant counterparts.

Methods for producing genetically modified organisms include both random and site-specific mutagenesis and transgenesis. Random methods take advantage of highly active or mutagenic substances such as chemicals, radiation or transposon insertional mutagenesis. Site-specific methods enable precise engineering of genomes in living cells and organisms. Traditionally, site-specific mutagenesis has been carried out by using homologous recombination of an exogenous sequence which may be a gene, gene fragment or selectable marker at the DNA sequence desired for modification.

Alternative site-specific genome modification technologies, including nucleases and homing endonucleases have been developed, such as Xanthomonas TAL Nucleases (XTNs). The site-specific technologies, such as XTNs, can be modified in order to specifically bind to sites within the genome of many organisms. XTNs may be used to introduce targeted double-stranded or single-stranded breaks in the DNA, which can lead to small deletions at the site of the break during the Non-Homologous End Joining (NHEJ) process, thereby producing gene knockouts in cells and organisms. XTNs can also generate breaks in the DNA which can increase the frequency of exogenous sequence introduction by homologous recombination, thereby enabling specific gene editing (e.g.—correction or mutation) or producing gene knock-ins in cells and organisms.

XTNs previously have not been used to produce site-specific genetic modifications in spermatogonial stem cells (SSCs) derived from rats, or from SSCs derived from many other agriculturally or biomedically important species. Additionally, XTNs have not been used to create site-specific mutations in other types of stem cells, such as embryonic stem (ES) cells, induced pluripotent stem cells (iPS), somatic stem cells derived from many other agriculturally or biomedically important species. Stem cells containing site-specific mutations can be used to rapidly and cost-effectively generate genetically modified organisms. While targeted mutations have been described in many somatic cells and cell lines and in embryonic stem cells from a few species, SSCs from rats and most other species have not been successfully targeted using site-specific technologies.

Thus, there remains a need for compositions and methods for generating site-specific mutations in stem cells that can be used to produce genetic modifications in rats and other agriculturally or biomedically important species.

### SUMMARY OF THE INVENTION

In accordance with the purposes of this invention, as embodied and broadly described herein, this invention relates to methods for site-specific genetic engineering using Xanthomonas TAL Nucleases (XTNs) of stem cells and gametes, including but not limited to pluripotent cells, totipotent cells, somatic stem cells, spermatogonial stem cells (SSCs), embryonic stem (ES) cells, induced pluripotent stem (iPS) cells, embryos, germ cells, primordial germ cells (PGCs), plant tube cells, pollen cells, and spores. Methods for site-specific engineering of stem cells include, but are not limited to using site specific DNA binding and cleaving proteins such as XTNs.

Site-specific engineering of stem cells results in altered function of gene(s) or gene product(s) and genetically modified organisms, and cell or tissue culture models are produced from these engineered stem cells. Modified stem cells and organisms include knockout and knockin cells and organisms.

In another aspect, the invention relates to genetically modified organisms created by site-specific engineering using XTNs including but not limited to mammals, including rats, mice, pigs, rabbits, guinea pigs, dogs, non-human primates, mini-pigs, as well as plants, including but not limited to maize, soybean, rice, potato, wheat, tobacco, tomato, and Arabidopsis, as well as the descendants and ancestors of such organisms.

In another embodiment, the invention provides kits that are used to produce site specific-mutations in stem cells, which can be used to generate genetically modified organisms. The kits typically include one or more site-specific genetic engineering technology, such as XTNs. The kit may also comprise one or more sets of stem cells for site-specific modification. In some embodiments of the invention, the stem cells may include, but are not limited to, spermatogonial stem cells (SSCs), as well as media and conditions necessary for growing SSCs. In some embodiments, the kit comprise exogenous sequences for site-specific genomic introduction, such as but not limited to reporter genes or selectable markers. In some embodiments, the kit comprises instructions for (i) introducing the XTNs into the stem cells (ii) identifying stem cells which have been site specifically modified by the XTN (iii) growing site-specifically modified stem cells in media or conditions necessary and to numbers required for stem cells to produce genetically modified organisms or effect germline transmission in an animal; (iv) using or transplanting the grown stem cells to produce a genetically modified organism; and/or (v) identifying which organisms or progeny comprise the site-specific mutation of interest.

In some embodiments of the invention, a composition comprises one or more stem cells or one or more embryos, the one or more stem cells or one or more embryos comprise one or more of the following mutations: (i) a deletion mutation; (ii) a knockout mutation; and/or (iii) an addition of a heterologous nucleic acid sequence; the one or more mutations of (i), (ii), and/or (iii) are site-specific mutations caused by a Xanthomonas TAL Nucleases (XTN).

In some embodiments of the invention, the heterologous nucleic acid sequence is chosen from a selectable marker or an orthologous gene.

In some embodiments of the invention, the one or more stem cells is chosen from a spermatogonial stem cell (SSC), an embryonic stem cell, or an induced pluripotent stem cell.

In some embodiments of the invention, the one or more stem cells is derived from the germline lineage of an animal or plant.

In some embodiments of the invention, the one or more stem cells or the one or more embryos further comprise at least one inverted tandem repeat of a transposon or a variant thereof.

In some embodiments of the invention, the one or more stem cells is a somatic stem cell.

In some embodiments of the invention, an organism comprising one or more stem cells, the one or more stem cells comprise one or more of the following mutations: (i) a deletion mutation; (ii) a knockout mutation; and/or (iii) an addition of a heterologous nucleic acid sequence; the one or more mutations of (i), (ii), and/or (iii) are site-specific mutations caused by a XTN.

In some embodiments of the invention, the one or more stem cells comprises an SSC.

In some embodiments of the invention, the one or more stem cells further comprise at least one inverted tandem repeat of a transposon or variant thereof.

In some embodiments of the invention, a composition comprising one or more stem cells or one or more embryos and: (a) a XTN that cleaves a nucleic acid sequence at a pre-determined location within the genome of the one or more stem cells or the one or more embryos; or (b) a nucleic acid sequence that encodes a XTN that cleaves a nucleic acid of the stem cell at a pre-determined site within the genome of the stem cell or the embryo; the one or more stem cells is derived from the germline lineage of an animal or plant.

In some embodiments of the invention, the stem cell is a spermatogonial stem cell derived from a rat or mini pig.

In some embodiments of the invention, the one or more stem cells or the one or more embryos further comprise at least one inverted tandem repeat of a transposon or a variant thereof.

In some embodiments of the invention, the one or more stem cells or the one or more embryos further comprise: (a) one or more nucleic acid sequences at least 70% homologous to a nucleic acid sequence chosen from:

(a)
(SEQ ID NO: 1)
CAGTTGAAGTCGGAAGTTTACATACACTTAAGTTGGAGTCATTAAAACT
CGTTTTTCAACTACTCCACAAATTTCTTGTTAACAAACAATAGTTTTGG
CAAGTCAGTTAGGACATCTACTTTGTGCATGACACAAGTCATTTTTCCA
ACAATTGTTTACAGACAGATTATTTCACTTATAATTCACTGTATCACAA
TTCCAGTGGGTCAGAAGTTTACATACACTAAGT;
(b)
(SEQ ID NO: 2)
ATTGAGTGTATGTAAACTTCTGACCCACTGGGAATGTGATGAAAGAAAT
AAAAGCTGAAATGAATCATTCTCTCTACTATTATTCTGATATTTCACAT
TCTTAAAATAAAGTGGTGATCCTAACTGACCTAAGACAGGGAATTTTTA
CTAGGATTAAATGTCAGGAATTGTGAAAAAGTGAGTTTAAATGTATTTG
GCTAAGGTGTATGTAAACTTCCGACTTCAACTG;
(c)
(SEQ ID NO: 3)
CCCTAGAAAGATAGTCTGCGTAAAATTGACGCATGCATTCTTGAAATAT
TGCTCTCTCTTTCTAAATAGCGCGAATCCGTCGCTGTGCATTTAGGACA
TCTCAGTCGCCGCTTGGAGCTCCCGTGAGGCGTGCTTGTCAATGCGGTA
AGTGTCACTGATTTTGAACTATAACGACCGCGTGAGTCAAAATGACGCA
TGATTATCTTTTACGTGACTTTTAAGATTTAACTCATACGATAATTATA
TTGTTATTTCATGTTCTACTTACGTGATAACTTATTATATATATATTTT
CTTGTTATAGATATC;
and
(d)
(SEQ ID NO: 4)
TAAAAGTTTTGTTACTTTATAGAAGAAATTTTGAGTTTTTGTTTTTTTT
TAATAAATAAATAAACATAAATAAATTGTTTGTTGAATTTATTATTAGT
ATGTAAGTGTAAATATAATAAAACTTAATATCTATTCAAATTAATAAAT
AAACCTCGATATACAGACCGATAAAACACATGCGTCAATTTTACGCATG
ATTATCTTTAACGTACGTCACAATATGATTATCTTTCTAGGG;

or (b) a fragment of a nucleic acid sequence 70% homologous to SEQ ID NO:1, SEQ ID NO:2, SEQ ID NO:3, and SEQ ID NO:4.

In some embodiments of the invention, a composition comprising one or more progeny of the organism, the one or more progeny comprise any one or more of the one or more mutations (i), (ii), and (iii). In some embodiments of the invention, the one or more progeny further comprise at least one inverted tandem repeat of a transposon or variant thereof. In some embodiments of the invention, the composition is a colony of mammals. In some embodiments of the invention, the composition comprises a single organism that is from the first generation of an organism grown or derived from the stem cell or embryo.

In some embodiments of the invention, the organism is a plant or animal.

In some embodiments of the invention, the organism is a mini pig.

In some embodiments of the invention, the organism is a rat or mouse.

In some embodiments of the invention, the organism is chosen from a mouse, pig, rabbit, dog, cat, goat, non-human primate, mini pig, ferret, farm animals, fish, chicken, and bird.

In some embodiments of the invention, the organism is a plant chosen from: rice, tobacco, wheat, potato, soybean, tomato, Arabidopsis, maize. In some embodiments of the invention, the organism is chosen from a salmonoid, carp, tilopia, or tuna.

In some embodiments of the invention, the organism is an insect.

In some embodiments of the invention, a mammal comprising one or more stem cells derived from the germline lineage of an animal, the one or more stem cells comprise one or more of the following mutations: (i) a deletion mutation; (ii) a knockout mutation; and/or (iii) an addition of a heterologous nucleic acid sequence; the one or more mutations of (i), (ii), and/or (iii) are site-specific mutations caused by a XTN.

In some embodiments of the invention, the one or more stem cells are transplanted from an in vitro culture. In some embodiments of the invention, the mammal further comprises a nucleic acid that comprises a transposon sequence that is at least 70% homologous to: SEQ ID NO:1, SEQ ID NO:2, SEQ ID NO:3, and/or SEQ ID NO:4. In some embodiments of the invention, the mammal further comprises a nucleic acid that comprises a transposon sequence that is at least 70% homologous to: SEQ ID NO:1, SEQ ID NO:2, SEQ ID NO:3, and/or SEQ ID NO:4. In some embodiments of the invention, the mammal further comprises a nucleic acid that comprises a transposon sequence that is at least 75% homologous to: SEQ ID NO:1, SEQ ID NO:2, SEQ ID NO:3, and/or SEQ ID NO:4. In some embodiments of the invention, the mammal further comprises a nucleic acid that comprises a transposon sequence that is at least 80% homologous to: SEQ ID NO:1, SEQ ID NO:2, SEQ ID NO:3, and/or SEQ ID NO:4. In some embodiments of the invention, the mammal further comprises a nucleic acid that comprises a transposon sequence that is at least 85% homologous to: SEQ ID NO:1, SEQ ID NO:2, SEQ ID NO:3, and/or SEQ ID NO:4. In some embodiments of the invention, the mammal further comprises a nucleic acid that comprises a transposon sequence that is at least 90% homologous to: SEQ ID NO:1, SEQ ID NO:2, SEQ ID NO:3, and/or SEQ ID NO:4. In some embodiments of the invention, the mammal further comprises a nucleic acid that comprises a transposon sequence that is at least 95% homologous to: SEQ ID NO:1, SEQ ID NO:2, SEQ ID NO:3, and/or SEQ ID NO:4. In some embodiments of the invention, the mammal further comprises a nucleic acid that comprises a transposon sequence that is at least 96% homologous to: SEQ ID NO:1, SEQ ID NO:2, SEQ ID NO:3, and/or SEQ ID NO:4. In some embodiments of the invention, the mammal further comprises a nucleic acid that comprises a transposon sequence that is at least 97% homologous to: SEQ ID NO:1, SEQ ID NO:2, SEQ ID NO:3, and/or SEQ ID NO:4. In some embodiments of the invention, the mammal further comprises a nucleic acid that comprises a transposon sequence that is at least 98% homologous to: SEQ ID NO:1, SEQ ID NO:2, SEQ ID NO:3, and/or SEQ ID NO:4. In some embodiments of the invention, the mammal further comprises a nucleic acid that comprises a transposon sequence that is at least 99% homologous to: SEQ ID NO:1, SEQ ID NO:2, SEQ ID NO:3, and/or SEQ ID NO:4. In some embodiments of the invention, the mammal further comprises a nucleic acid that comprises a transposon sequence that is no more than 70% homologous to: SEQ ID NO:1, SEQ ID NO:2, SEQ ID NO:3, and/or SEQ ID NO:4.

In some embodiments of the invention, the mammal further comprises a nucleic acid that comprises a transposon sequence that is no more than 75% homologous to: SEQ ID NO:1, SEQ ID NO:2, SEQ ID NO:3, and/or SEQ ID NO:4.

In some embodiments of the invention, the mammal further comprises a nucleic acid that comprises a transposon sequence that is no more than 80% homologous to: SEQ ID NO:1, SEQ ID NO:2, SEQ ID NO:3, and/or SEQ ID NO:4. In some embodiments of the invention, the mammal further comprises a nucleic acid that comprises a transposon sequence that is no more than 85% homologous to: SEQ ID NO:1, SEQ ID NO:2, SEQ ID NO:3, and/or SEQ ID NO:4.

In some embodiments of the invention, the mammal further comprises a nucleic acid that comprises a transposon sequence that is no more than 90% homologous to: SEQ ID NO:1, SEQ ID NO:2, SEQ ID NO:3, and/or SEQ ID NO:4. In some embodiments of the invention, the mammal further comprises a nucleic acid that comprises a transposon sequence that is no more than 95% homologous to: SEQ ID NO:1, SEQ ID NO:2, SEQ ID NO:3, and/or SEQ ID NO:4.

In some embodiments of the invention, the one or more stem cells are spermatogonial stem cells.

In some embodiments of the invention, the mammal is a rat or mini pig. In some embodiments of the invention, the mammal is a sterile male rat or sterile male mini pig. In some embodiments of the invention, the rat or mini pig is DAZL deficient or DAZL−/−. In some embodiments of the invention, a colony of genetically modified organisms comprises:

at least one organism comprising one or more stem cells, the one or more stem cells comprise one or more of the following mutations: (i) a deletion mutation; (ii) a knockout mutation; and/or (iii) an addition of a heterologous nucleic acid sequence; the one or more mutations of (i), (ii), and/or (iii) are site-specific mutations caused by a XTN; and (b) progeny of the organism of subpart (a).

In some embodiments of the invention, the heterologous nucleic acid is a selectable marker or an orthologous gene.

In some embodiments of the invention, the at least one organism and the progeny further comprise at least one inverted tandem repeat of a transposon or variant thereof.

In some embodiments of the invention, the at least one organism and the progeny further comprise a nucleic acid that comprises a transposon sequence that is at least 70% homologous to: SEQ ID NO:1, SEQ ID NO:2, SEQ ID NO:3, and/or SEQ ID NO:4.

In some embodiments of the invention, the invention relates to a method of generating one or more genetically modified organisms comprising:

(a) contacting at least one stem cell derived from the germline lineage of an animal or plant by the stem cell with: (i) at least one XTN that mutates a gene of interest; or (ii) at least one expression vector that encodes a XTN that mutates a gene of interest, thereby creating at least one stem cell comprising at least one mutation at a gene of interest;

(b) expanding an in vitro culture of the at least one stem cell comprising at least one mutation at a gene of interest;

(c) implanting one or more stem cells from the culture of step (b) into an organism.

In some embodiments of the invention, the invention relates to a method of generating one or more genetically modified organisms comprising:

(a) contacting at least a first and second set of stem cell derived from the germline lineage of an animal or plant with: (i) at least one XTN that mutates a gene of interest; or (ii) at least one expression vector that encodes a XTN that mutates a gene of interest, thereby creating at least a first and second set of stem cells comprising at least one mutation at a gene of interest;

(b) expanding an in vitro culture of the at least one stem cell comprising at least one mutation at a gene of interest;

(c) implanting one or more sets of stem cells from the culture of step (b) into an organism.

In some embodiments, the method further comprises a third, fourth, fifth, sixth, seventh, eighth, ninth, or ten or more sets of stem cells which have been mutated in a site-specific fashion by a XTN, and, in which case, after expanding each of the third, fourth, fifth, sixth, seventh, eighth, ninth, or ten or more sets of mutated stem cells, each set of transplanted into a single organism.

In some embodiments, the single organism that comprises a set of mutated stem cells is a sterile male.

In some embodiments of the invention, the organism is capable of passing at least one mutation at a gene of interest to progeny by germline transmission.

In some embodiments of the invention, the genetically modified organism is a mammal.

In some embodiments of the invention, the genetically modified organism is a rat or mini pig.

In some embodiments of the invention, the genetically modified organism is a sterile male rat or sterile male mini pig.

In some embodiments of the invention, the method further comprises: breeding the organism implanted with the one or more stem cells with another animal to generate one or more progeny that comprise the mutated gene of interest. In some embodiments of the invention, the method further comprises: breeding the organism implanted with the one or more set of stem cells with another animal to generate one or more progeny that comprise the one or more mutated genes of interest that correspond to each of the mutated stem cell lines.

In some embodiments of the invention, the progeny are mammals.

In some embodiments of the invention, a method of breeding a colony of genetically modified organisms comprising:

(a) contacting at least one stem cell derived from the germline lineage of an animal or plant by the stem cell with: (i) at least one XTN that mutates a gene of interest; or (ii) at least one expression vector that encodes a XTN that mutates a gene of interest, thereby creating a stem cell comprising at least one mutation at a gene of interest;

(b) expanding an in vitro culture of the stem cell comprising at least one mutation at a gene of interest;

(c) implanting the at least one stem cell comprising at least one mutation at a gene of interest from the culture of step (b) into a first organism.

(d) breeding the first organism with a second organism of the same species;

(e) selecting progeny of the first and second organism that comprise the at least one mutation at a gene of interest; and

(f) breeding the progeny to create a colony of organisms that comprise the at least one mutation at a gene of interest.

In some embodiments of the invention, the first and second organisms are mammals.

In some embodiments of the invention, the first and second organisms are rats or mini pigs.

In some embodiments of the invention, the invention relates to a method of manufacturing a first filial generation of genetically modified organisms comprising two or more distinct subsets of organisms, the method comprising:

(a) contacting a first stem cell with: (i) a XTN that mutates a first gene of interest; or (ii) an expression vector that encodes a XTN that mutates a first gene of interest; thereby creating a first stem cell comprising a first mutation;

(b) contacting a second stem cell with a modifying agent, thereby creating a second stem cell comprising a second mutation;

(c) expanding an in vitro culture of each of the first and the second stem cells;

(d) implanting a mixed population of stem cells comprising the first and the second stem cells into an organism;

(e) breeding the organism with another organism of the same species.

In some embodiments of the invention, the first filial generation of genetically modified organisms comprises two or more sets of organisms, each set comprising a distinct mutation of interest derived from a haplotype of distinct stem cells transplanted into a parent of the organism.

In some embodiments of the invention, at least one stem cell of the mixed population is a spermatogonial stem cell of a mammal.

In some embodiments of the invention, the organism is a mammal.

In some embodiments of the invention, a kit comprising:

(a) a XTN or a nucleic acid sequence that encodes a XTN that cleaves a nucleic acid sequence at a gene of interest; and

(b) an instruction manual comprising directions; and, optionally

In some embodiments of the invention, a kit comprising:

(a) In some embodiments of the invention; and, optionally

(b) culture media for the one or more stem cells or one or more embryos.

In some embodiments of the invention, the kit comprises:

(a) an XTN or a nucleic acid sequence that encodes an XTN that cleaves a nucleic acid sequence at a gene of interest; and optionally

(b) culture media for the one or more stem cells or one or more embryos.

In some embodiments of the invention, the kit comprises:

(a) an XTN or a nucleic acid sequence that encodes an XTN that cleaves a nucleic acid sequence at a gene of interest; and

(b) one or more stem cell lines derived from a germline lineage of animal or plant; and, optionally

(c) culture media for the one or more stem cells or one or more embryos; and, optionally

(d) an instruction manual that comprises instructions on how to mutate the one or more stem cells with the XTN or a nucleic acid sequence that encodes the XTN that cleaves a nucleic acid sequence at a gene of interest.

Additional advantages of the invention will be set forth in part in the description which follows, and in part will be obvious from the description, or may be learned by practice of the invention.

The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWING

This invention, as defined in the claims, can be better understood with reference to the following drawings:

FIG. 1 depicts a schematic of spermatogonial stem cells (SSCs) separated into multiple colonies will be genetically modified with different XTNs. The genetically modified SSCs will be selected and pooled together to form a pool of SSCs containing different genetic modifications relating to the production of different genetically modified organisms using a single recipient male.

FIG. 2 depicts a schematic of a colony of wild type rat spermatogonial stem cells (SSCs) shown in cell culture.

FIG. 3 depicts a schematic of propagation of SSCs in cell culture

FIG. 4 depicts a schematic of transfection of SSCs with XTN and fluorescent marker constructs.

FIG. 5 depicts a Xanthomonas TAL Nucleases (XTN) recognition sites for the rat Rag1 gene. Several binding and mutation sites are shown highlighted in blue, green and purple.

FIG. 6 depicts a schematic for transplantation of genetically modified rat SSC transplantation into sterile recipient male rats. The genetically modified SSCs will be used to produce genetically modified rats by mating recipient males with wild type (WT) females.

FIG. 7 depicts the detailed description of the minipig Rag1 sequence and proposed XTN binding and mutation site.

FIG. 8 depicts the location and sequence identity of XTN-created SSC clones.

FIG. 9 depicts the sequence identity of homing endonuclease-created clones of embryonic stem cells.

### DETAILED DESCRIPTION OF THE INVENTION

Described herein are methods for site-specific genetic modification of stem cells which may be used to produce genetically modified organisms. Site-specific genetic modification includes but is not limited to mutations that cause deletions or knockout mutations, as well as mutations that can produce insertions or knockin mutations. In one embodiment, the invention provides site-specific modification of stem cells, especially spermatogonial stem cells (SSCs). The embodiment includes the site-specific technologies, especially Xanthomonas TAL Nucleases (XTNs). In another embodiment the genetically modified stem cells are somatic stem cells, embryonic stem (ES) cells, induced pluripotent stem (iPS) cells, embryos and other gametes or germ cells and the site-specific technologies include Xanthomonas TAL Nucleases (XTNs). Also described are methods for identifying cells that have acquired site-specific modifications and generating genetically modified organisms from genetifcally modified stem cells. In one embodiment, the invention includes methods for the use of spermatogonia or spermatogonial stem cells (SSCs) containing site-specific genetic modifications are expanded and grown to adequate numbers and transplanted into azoospermic recipient males that are genetically or chemically sterile. In another embodiment, ES cells, iPS cell-, and embryos or other gametes are used to produce organisms containing site-specific mutations.

Definitions

The present invention may be understood more readily by reference to the following detailed description of preferred embodiments of the invention and the Examples included therein and to the Figures and their previous and following description. Although any methods and materials similar or equivalent to those described herein can be used in the practice or testing of the present invention, the preferred methods, devices, and materials are now described. All references, publications, patents, patent applications, and commercial materials mentioned herein are incorporated by reference in their entirety for the purpose of describing and disclosing the materials and/or methodologies which are reported in the publications which might be used in connection with the invention. Nothing herein is to be construed as an admission that the invention is not entitled to antedate such disclosure by virtue of prior invention.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Throughout this application, reference is made to various proteins and nucleic acids. It is understood that any names used for proteins or nucleic acids are art-recognized names, such that the reference to the name constitutes a disclosure of the molecule itself.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

“Addition of heterologous sequence” is meant to be any introduction of deoxyribonucleotide, nucleotide or DNA sequence within a gene, chromosome or genome of an organism. Also known as a “knock-in” which is meant an alteration in the nucleic acid sequence that replaces the endogenous, normal or wild-type allele with an exogenous allele. The exogenous allele includes but is not limited to a full length gene of the same or a different species, a section of a gene of the same or different species, a replacement cassette and reporter or selection genes and markers. Knock-in mutations can be produced by homologous recombination, site-specific deletion, repair mechanism provocation via targeting proteins, as well as site specific targeted DNA transposons.

A “coding sequence” or a sequence “encoding” an expression product, such as a RNA, polypeptide, protein, or enzyme, is a nucleotide sequence that, when expressed or translated, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

“Complementary,” as used herein, refers to the subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs).

“DAZL deficient organisms” or “DAZL deficient rats” or “DAZL−/−” or “DAZL knockdown” means male organisms which have a lack of proper function in Deleted In-Azoospermia (DAZL) genes. In some embodiments, DAZL deficient organisms fail to produce mature haploid gametes. In some embodiments, DAZL deficient organisms are infertile. In some embodiment, DAZL deficient organisms do not express DAZL genes. In some embodiments, the DAZL-deficient organisms express defective or only diminimis amounts of DAZL genes which do not produce sufficient amounts of bioactive protein necessary to produce mature haploid gametes.

A “deletion mutation” means a type of mutation that involves the loss of genetic material, deoxyribonucleotide, nucleotide, DNA, gene or chromosome which may be from a single base to an entire piece of chromosome. Deletion of one or more nucleotides in the DNA, may relate to an altered reading frame or non-reading frame of the gene, chromosome or genome; hence, it could result in a complete absence of the synthesis, synthesis of a nonfunctional, or synthesis followed be degradation of DNA, RNA, peptide, polypeptide or protein.

The terms “derived from the germline lineage of an animal or plant” mean (as to any animal, cell, tissues or biomaterial—including nucleotides or DNA or genes or chromosomes or genomes or transgenes or mutations that may be passed on to offspring) obtained or originating from the germ cells of a animal or plant. In some embodiments, the one or more progeny of a parent line may contain a mutated gene of interest which originated from the haplotype of a stem cell transplanted into the testes of the parent line. In such an example the mutated gene of interest is derived from the germline of the parent line.

“Disease state” is a condition of an organism, tissue or tissues, or cells that exhibit unknown or abnormal clinical features, characteristics, or phenotypes. In some embodiments, the condition relates to an organism, tissue or tissues, or cells, that exhibit a phenotype associated with a known disease. The disease state may be characterized by clinical features such as hyperinsulinemia in diabetes or diagnostic features such as biomarker association as well as genetic features such as mutations and polymorphisms.

“Embryo” is a multicellular diploid eukaryote in early stage of development.

“Embryonic like cells from umbilical cord blood” or “CBEs” are cells that can be isolated from umbilical cord blood that have embryonic like properties such as the ability to differentiate into multiple germ layers and expression of embryonic markers.

“Embryonic stem cell” or ES cell is a pluripotent cell derived from the inner mass of the blastocyst or early stage embryo.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed”. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “gene”, also called a “structural gene,” means a DNA sequence that codes for or corresponds to a particular sequence of amino acids which comprise all or part of one or more proteins or enzymes, and may or may not include introns and regulatory DNA sequences, such as promoter sequences, 5′-untranslated region, or 3′-untranslated region which affect for example the conditions under which the gene is expressed. Some genes, which are not structural genes, may be transcribed from DNA to RNA, but are not translated into an amino acid sequence. Other genes may function as regulators of structural genes or as regulators of DNA transcription.

“Gene of interest” refers to a nucleotide, nucleotide sequence, DNA, RNA, polypeptide, sequence on a chromosome or within the genome of an organism which is to be genetically modified or altered in some way. The gene of interest can be mutated or its nucleotide sequence may be altered.

“Genetic background” or “strain” refers to a genetic composition that is characteristic of an organism. Organisms that have been bred may have a known genetic strain that may be useful for different research reasons. Organisms that evolve in regions of the earth contain different genetic backgrounds which may alter gene function and important physiological functions.

“Genetic modification associated with the gene of interest” means a mutation or other genetic modification which corresponds to a gene that is being studied or selected for. The genetic modification may involve either endogenous or exogenous genes.

“Genetically modified” or “genetic modification” means a gene or other DNA sequence that is altered from its native state (e.g. by insertion mutation, deletion mutation, nucleic acid sequence mutation, or other mutation), or that a gene product is altered from its natural state (e.g. by delivery of a transgene that works in trans on a gene's encoded mRNA or protein, such as delivery of inhibitory RNA or delivery of a dominant negative transgene). “Mutations” may produce organisms that are genetically modified or a specific genetic modification. “Mutations” may include but are not limited to one or more nucleic acid substitutions, deletions, frameshift mutations, or nonsense mutations.

A “germ cell” is a cell that gives rise to the gametes of an organism. The germ cell is often a pluripotent cell which can differentiate into gametes as well as other biological cell types. A germ cell includes but is not limited to pluripotent cells, totipotent cells, spermatogonial stem cells (SSCs), embryonic stem (ES) cells, induced pluripotent stem (iPS) cells, embryos, germ cells, primordial germ cells (PGCs), plant tube cells, pollen cells, and spores.

By “exon” is meant a region of a gene which includes sequences which are used to encode the amino acid sequence of the gene product.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is such an element operatively associated with a different gene than the one it is operatively associated with in nature.

As used herein, the term “homology” refers to the subunit sequence identity or similarity between two polymeric molecules e.g., between two nucleic acid molecules, e.g., between two DNA molecules, or two polypeptide molecules. When a subunit position in both of the two molecules is occupied by the same monomeric subunit, e.g., if a position in each of two polypeptide molecules is occupied by phenylalanine, then they are identical at that position. The homology between two sequences, most clearly defined as the % identity, is a direct function of the number of identical positions, e.g., if half (e.g., 5 positions in a polymer 10 subunits in length) of the positions in two polypeptide sequences are identical then the two sequences are 50% identical; if 70% of the positions, e.g., 7 out of 10, are matched or homologous, the two sequences share 70% identity. By way of example, the polypeptide sequences ACDEFG and ACDHIK share 50% identity and the nucleotide sequences CAATCG and CAAGAC share 50% identity.

“Homologous recombination” is the physical exchange of DNA expedited by the breakage and reunion of two non-sister chromatids. In order to undergo recombination the DNA duplexes must have complimentarity. The molecular mechanism is as follows: DNA duplexes pair, homologous strands are nicked, and broken strands exchange DNA between duplexes. The region at the site of recombination is called the hybrid DNA or heteroduplex DNA. Second nicks are made in the other strand, and the second strand crosses over between duplexes. After this second crossover event the reciprocal recombinant or splice recombinant is created. The duplex of one DNA parent is covalently linked to the duplex of another DNA parent. Homologous recombination creates a stretch of heteroduplex DNA.

A “induced pluripotent stem cell” or (iPS) cell is an adult cell that has been reprogrammed back to an embryonic like state. iPS cells can differentiate into many different cell types as well as produce genetically modified organisms.

The term “insertional mutation” is used herein to refer the translocation of nucleic acid from one location to another location which is in the genome of an organism so that it is integrated into the genome, thereby creating a mutation in the genome. Insertional mutations can also include knocking out or knocking in of endogenous or exogenous DNA via gene trap, cassette insertion, provoking deletion of a targeted locus followed by recombination. Exogenous DNA can access the cell via electroporation or chemical transformation. If the exogenous DNA has homology with chromosomal DNA it will align itself with endogenous DNA. The exogenous DNA is then inserted or disrupts the endogenous DNA via two adjacent crossing over events, known as homologous recombination. A targeting vector can use homologous recombination for insertional mutagenesis. Insertional mutagenesis of endogenous or exogenous DNA can also be carried out via DNA transposon, and deletion by site specific targeting proteins followed by homologous recombination. The DNA transposon is a mobile element that can insert itself along with additional exogenous DNA into the genome. Insertional mutagenesis of endogenous or exogenous DNA can be carried out by retroviruses. Retroviruses have a RNA viral genome that is converted into DNA by reverse transcriptase in the cytoplasm of the infected cell. Linear retroviral DNA is transported into the nucleus, and become integrated by an enzyme called integrase. Insertional mutagenesis of endogenous or exogenous DNA can also be done by retrotransposons in which an RNA intermediate is translated into double stranded DNA by reverse transcriptase, and inserting itself into the genome.

The term “knockout” means a mutation or an alteration in the nucleic acid sequence that reduces the biological activity of a peptide, polypeptide, protein, or RNA normally encoded therefrom by at least 80% compared to the unaltered gene. The alteration may be an insertion, deletion, frameshift mutation, or missense mutation. The alteration may be an insertion or deletion, or is a frameshift mutation that creates a stop codon. The knockout mutation may result in complete elimination of the function of a gene or nucleotide sequence. A knockout mutation may also be known as a null mutation.

“Minipig” or “pig” are breeds of inbred or outbred swine which can be used for research.

A “modifying agent” or “mutagen” is meant to be a physical or biological or chemical agent that changes genetic material or nucleotides, DNA, genes, chromosomes, genomes or organisms. Modifying agents can include natural and engineered proteins such as XTNs.

A “mutation” is a change or in the process of change. A change in the genetic material in the organism, which is transmitted to the organism's progeny. A permanent or heritable change in a nucleotide sequence of a gene or chromosome; the process in which such a change occurs in a gene or chromosome. In some embodiments, a mutation involves the change in one or more deoxyribonucleotides, the modification being obtained by, for example, adding, deleting, inverting, or substituting nucleotides. Exemplary mutations include but are not limited to a deletion mutation, an insertion mutation, a non-sense mutation or a missense mutation. Thus, the terms “mutation” or “mutated” as used herein are intended to denote an alteration in the “normal” or “wild-type” nucleotide sequence of any nucleotide sequence or region of the allele. As used herein, the terms “normal” and “wild-type” are intended to be synonymous, and to denote any nucleotide sequence typically found in nature. The terms “mutated” and “normal” are thus defined relative to one another; where a cell has two chromosomal alleles of a gene that differ in nucleotide sequence, at least one of these alleles is a “mutant” allele as that term is used herein. A mutation may also be a “DNA” or “nucleic acid sequence mutation” or a “frameshift mutation”. Any mutation that alters a DNA sequence may cause one or more nucleic acid changes or deletions.

The terms “Non-homologous end joining (NHEJ)” refer to a cellular repair mechanism defined by the ligation of blunt ended double stand DNA breaks. The pathway is initiated by double strand breaks in the DNA, and works through the ligation of DNA duplex blunt ends. The first step is recognition of double strand breaks and formation of scaffold. The trimming, filling in of single stranded overhangs to create blunt ends and joining is executed by the NHEJ pathway. An example of NHEJ is repair of a DNA cleavage site created by a Xanthomonas TAL Nucleases (XTN) or zinc finger nuclease (ZFN). This would normally be expected to create a small deletion mutation.

“Nucleic Acid sequence mutation” is a mutation to the DNA that involves change of one or multiple nucleotides. A point mutation which affects a single nucleotide can result in a transition (purine to purine or pyrimidine to pyrimidine) or a transversion (purine to pyrimidine or pyrimidine to purine). A point mutation that changes a codon to represent a different amino acid is a missense mutation. Some point mutations can cause a change in amino acid so that there is a premature stop codon; these mutations are called nonsense mutations. A mutation that inserts or deletes a single base will change the entire downstream sequence and are known as frameshift mutations. Some mutations change a base pair but have no effect on amino acid representation; these are called silent mutations. Mutations to the nucleic acid of a gene can have different consequences based on their location (intron, exon, regulatory sequence, and splice joint).

As used herein, the term “phenotype” means any property of a cell or organism. A phenotype can simply be a change in expression of an mRNA or protein. Examples of phenotypes also include, but are in no way limited to, cellular, biochemical, histological, behavioral, or whole organismal properties that can be detected by the artisan. Phenotypes include, but are not limited to, cellular transformation, cell migration, cell morphology, cell activation, resistance or sensitivity to drugs or chemicals, resistance or sensitivity to pathogenic protein localization within the cell (e.g. translocation of a protein from the cytoplasm to the nucleus), profile of secreted or cell surface proteins, (e.g., bacterial or viral) infection, post-translational modifications, protein localization within the cell (e.g. translocation of a protein from the cytoplasm to the nucleus), profile of secreted or cell surface proteins, cell proliferation, signal transduction, metabolic defects or enhancements, transcriptional activity, cell or organ transcript profiles (e.g., as detected using gene chips), apoptosis resistance or sensitivity, animal behavior, organ histology, blood chemistry, biochemical activities, gross morphological properties, life span, tumor susceptibility, weight, height/length, immune function, organ function, any disease state, and other properties known in the art. In certain situations and therefore in certain embodiments of the invention, the effects of mutation of one or more genes in a cell or organism can be determined by observing a change in one or more given phenotypes (e.g., in one or more given structural or functional features such as one or more of the phenotypes indicated above) of the mutated cell or organism compared to the same structural or functional feature(s) in a corresponding wild-type or (non-mutated) cell or organism (e.g., a cell or organism that in which the gene(s) have not been mutated).

By “plasmid” is meant a circular strand of nucleic acid capable of autosomal replication in plasmid-carrying bacteria. The term includes nucleic acid which may be either DNA or RNA and may be single- or double-stranded. The plasmid of the definition may also include the sequences which correspond to a bacterial origin of replication.

“Pluripotent cells” are stem cells that are capable of differentiating into any of the germ layers and can produce any type of fetal and adult cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. The promoter may be operatively associated with other expression control sequences, including enhancer and repressor sequences.

The term “regulatory sequence” is defined herein as including promoters, enhancers and other expression control elements such as polyadenylation sequences, matrix attachment sites, insulator regions for expression of multiple genes on a single construct, ribosome entry/attachment sites, introns that are able to enhance expression, and silencers.

By “reporter gene” is meant any gene which encodes a product whose expression is detectable. A reporter gene product may have one of the following attributes, without restriction: fluorescence (e.g., green fluorescent protein), enzymatic activity (e.g., lacZ or luciferase), or an ability to be specifically bound by a second molecule (e.g., biotin or an antibody-recognizable epitope).

By “selectable marker” is meant a gene product which may be selected for or against using chemical compounds, especially drugs. Selectable markers often are enzymes with an ability to metabolize the toxic drugs into non-lethal products. For example, the pac (puromycin acetyl transferase) gene product can metabolize puromycin, the dhfr gene product can metabolize trimethoprim (tmp) and the bla gene product can metabolize ampicillin (amp). Selectable markers may convert a benign drug into a toxin. For example, the HSV tk gene product can change its substrate, FIAU, into a lethal substance. Another selectable marker is one which may be utilized in both prokaryotic and eukaryotic cells. The neo gene, for example, metabolizes and neutralizes the toxic effects of the prokaryotic drug, kanamycin, as well as the eukaryotic drug, G418.

By “selectable marker gene” as used herein is meant a gene or other expression cassette which encodes a protein which facilitates identification of cells into which the selectable marker gene is inserted.

A “site-specific mutation” is used herein to refer to a location in the genome that is predetermined as the position where a targeted mutation will take place. The site-specific mutation may result in a knockout, knock-in or otherwise genetically modified cell. It is also used herein to refer to a specific location in the genome that is modified by any insertion mutation or deletion mutation or nucleic acid sequence mutation or forced repair mutation.

“Somatic stem cell” or adult stem cell is a potent cell found in organs after embryonic development. Somatic stem cells can be isolated from organs and tissues and have the potential to differentiate into many cell types of that organ and organism.

“Spermatogonial stem cell” or (SSC) is meant to be a sperm stem cell which maintains spermatogenesis.

“Sterile” or “sterile animal” or “sterile male” is meant to be a animal which is unable to produce endogenous germ cells or is not capable of producing suitable numbers of endogenous germ cells or not capable of producing mature germ cells. Sterile animals may not be able to produce sperm or spermatids. Sterile animals may not be able to generate offspring or breed.

As used herein, the term “targeted genetic recombination” refers to a process wherein recombination occurs within a DNA target locus present in a host cell or host organism. Recombination can involve either homologous or non-homologous DNA.

“Totipotent cells” are cells that have the ability to divide and differentiate into any cell type including extraembryonic cells.

The term “transfection” means the introduction of a foreign nucleic acid into a cell. The term “transformation” means the introduction of a “foreign” (i.e. extrinsic or extracellular) gene, DNA or RNA sequence into a cell. In some embodiments, transformation means the introduction of a “foreign” (i.e. extrinsic or extracellular) gene, DNA or RNA sequence into an ES cell or pronucleus, so that the cell will express the introduced gene or sequence to produce a desired substance in an organism or genetically modified organism.

By “transgenic” is meant any organism which includes a nucleic acid sequence which is inserted by artifice into a cell and becomes a part of the genome of the organism that develops from that cell. Such a transgene may be partly or entirely heterologous to the transgenic organism. Although transgenic mice represent another embodiment of the invention, other transgenic mammals including, without limitation, transgenic rodents (for example, hamsters, guinea pigs, rabbits, and rats), and transgenic pigs, cattle, sheep, and goats are included in the definition.

A “variant” is a nucleotide, set of nucleotides, DNA, RNA, gene, chromosome, genome, cell or organism which differs. The variant may differ in nucleotide sequence, gene expression, RNA expression, protein expression and function, genotype, phenotype and characteristics. In some embodiments, the cells or embryos of the invention comprise variant transposon inverted tandem repeats (ITRs) that are at least 70, 75, 80, 85, 90, 95, 96, 97, 98, 99% homologous to known ITRs. In some embodiments, the variant is a variant of a transposon ITR shown in table 3.

The term “vector” is used interchangeably with the terms “construct”, “cloning vector” and “expression vector” and means the vehicle by which a DNA or RNA sequence (e.g. a foreign gene) can be introduced into a host cell, (e.g. ES cell or pronucleus) so as to transform the host and promote expression (e.g. transcription and translation) of the introduced sequence including but not limited to plasmid, phage, transposons, retrotransposons, viral vector, and retroviral vector. By “non-viral vector” is meant any vector that does not comprise a virus or retrovirus.

A “vector sequence” as used herein, refers to a sequence of DNA comprising at least one origin of DNA replication and at least one selectable marker gene.

Xanthomonas TAL nucleases or “XTN” or “XTNs” are nucleases that can be modified to specifically bind to sequences in the genome. XTNs are combined with nuclease for site specific cleavage.

The present invention provides methods to produce a desired site-specific mutation in a variety of stem cells in order to develop heterozygous or homozygous genetically modified organisms. In one embodiment, the method for producing the site-specific mutation is the use of a Xanthomonas TAL Nucleases (XTNs). Specifically, the invention pertains to a site-specific mutation generated in a stem cell, which includes but is not limited to somatic stem cells, spermatogonial stem cells (SSCs), embryonic stem (ES) cells, embryos, and induced pluripotent stem (iPS) cells. Stem cells with site-specific mutations are used to produce genetically modified organisms.

The methods of the present invention can be used to mutate any eukaryotic stem cell, including, but not limited to, haploid, diploid, triploid, tetraploid, or aneuploid. In one embodiment, the cell is diploid. Stem ells in which the methods of the present invention can be advantageously used include, but are not limited to stem cells such as somatic stem cells, SSCs, ES cells, iPS cells, embryos, or any cell capable of developing into organisms.

In one embodiment, the invention relates to a method to produce a site-specific knockout, knock-in or otherwise genetically modified stem cell. The site-specific mutation is generated using a XTN which cleaves the desired site, followed by NHEJ, resulting in deletion mutations. The site-specific mutation can be produced in spermatogonial stem cells (SSCs) which are used to generate heterozygous or homozygous genetically modified organisms.

In another embodiment, the invention relates to a method to produce a site-specific knockout, knock-in or otherwise genetically modified stem cell. The site-specific mutation is generated using a XTN which cleaves the desired site resulting in deletion mutations. The site specific mutation is produced in embryonic stem (ES) cells, which are used to generate heterozygous or homozygous genetically modified organisms.

In another embodiment, the invention comprises of methods to produce a site-specific knockout, knock-in or otherwise genetically modified stem cell. The site specific mutation is generated using a XTN which cleaves the desired site resulting in deletion mutations. The site-specific mutation is produced in induced pluripotent stem (iPS) cells, which are used to generate heterozygous or homozygous genetically modified organisms.

In another embodiment, the invention comprises of methods to produce a site-specific knockout, knockin or otherwise genetically modified stem cell. The site specific mutation is generated using a XTN which cleaves the desired site resulting in deletion mutations. The site-specific mutation is produced in embryos which are used to generate heterozygous or homozygous genetically modified organisms.

In certain embodiments of the invention, cells can be mutated within the organism or within the native environment as in tissue explants (e.g., in vivo or in situ). Alternatively, tissues or stem cells isolated from the organism using art-known methods and genes can be mutated according to the present methods. The tissues or stem cells are either maintained in culture (e.g., in vitro), or re-implanted into a tissue or organism (e.g., ex vivo). XTN nucleases

Xanthomonas TAL nucleases, referred to as XTNs from the plant Xanthomonas bind DNA sequences in a site-specific manner as a mechanism to regulate their genes. A central repeat domain containing multiple repeat units consisting of 33-35 amino acids determines nucleotide binding sites. Two essential adjacent amino acids known as repeat variable di-residue or RVDs are present in each repeat domain and separately specify a targeted base. The repeat domains and RVDs can be modified in order to target a gene or locus with high specificity (Mahfouz et a. (2011) PNAS 108, 6, 2623-2628). By fusing nuclease cleavage domains such as FokI to the XTNs, a nuclease is produced which is able to generate mutations in the genome of organisms in a site-specific manner. In one embodiment, XTNs are used to generate site specific mutations in SSCs or ES cells or iPS cells or embryos and other pluripotent cells.

XTN DNA binding specificity depends on the number and order of repeats in the DNA binding domain. Repeats are generally composed of 34-35 amino acids. Nucleotide binding specificity is determined by the 12 and 13 amino acids, called the repeat variable diresidue (RVD), within the DNA binding domain repeats. The RVDs bind to one or more nucleotides and the code has been deciphered using arbitrary RVDs as follows: asaparagine/isoleucine (NI)=A; histidine/aspartic acid (HD)=C; asparagine/glycine (NG)=T; asparagines/asparagines (NN)=A, G; asparagines/serine (NS)=A, C, G and T. Since the RVD binding code is deciphered, natural or codon-optimized versions of natural XTNs can be used as a scaffold to generate sequence specific DNA binding XTNs. The repeats and RVDs in the DNA binding domains of XTNs may be modified and synthesized to generate site specific DNA binding XTNs. The DNA cleavage domain of nucleases are fused into the XTN to produce a hybrid XTN which binds to a specific site on the DNA and produces mutations.

Genetic modification of SSCs using XTNs requires undifferentiated SSCs, transfection of the SSCs with XTNs and a selection marker, clonal selection of genetically modified SSCs, germline transmission of genetically modified SSCs, and germline transmission of recipient founders.

In some embodiment of the invention, genetic modification of SSCs using XTNs relates to generating mutations at higher efficiency due to the unique nature of SSCs, including but not limited to chromatin structure and methylation patterns.

In some embodiment of the invention, genetic modification of SSCs using XTNs relates to generating multiple mutations in the same SSC or SSC line, which relates to generating genetically modified organisms with multiple mutations in fewer experimental steps and in a shorter timeframe than is possible with other systems.

In some embodiment of the invention, genetic modification of SSCs using XTNs relates to generating multiple mutations in the same SSC or SSC line in multiple and consecutive experiments or transfections, which relates to generating genetically modified organisms with multiple mutations in fewer experimental steps and in a shorter timeframe than is possible with other systems.

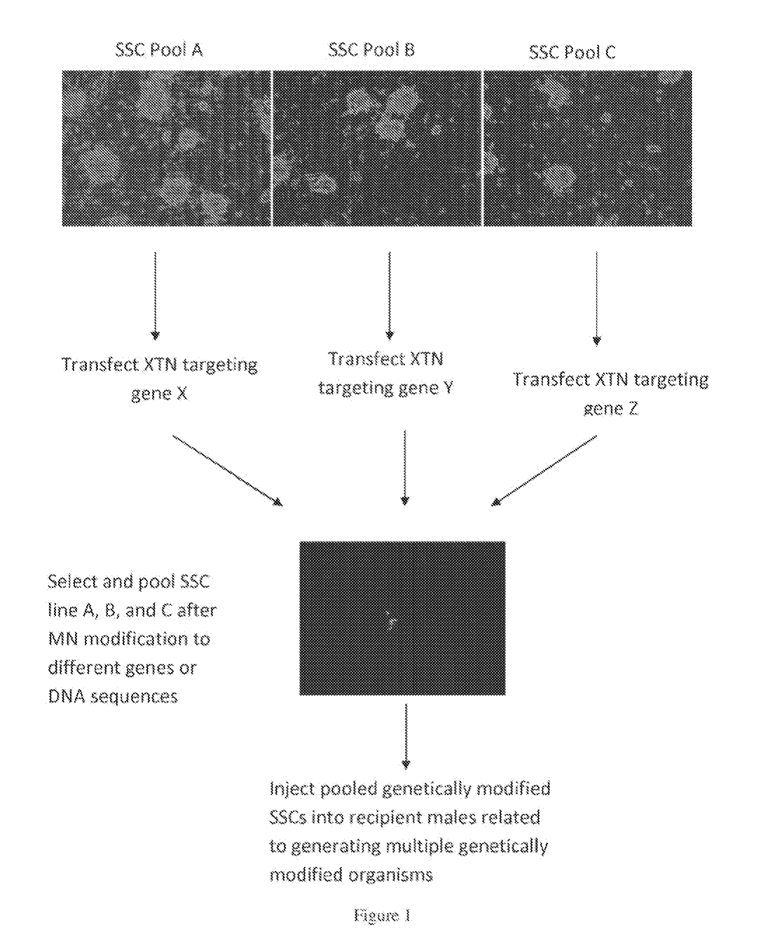

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be two or more.

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be three or more.

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be four or more.

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be five or more.

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be six or more.

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be seven or more.

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be eight or more.

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be nine or more.

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be ten or more.

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be eleven or more.

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be twelve or more.

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be thirteen or more.

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be fourteen or more.

In some embodiment of the invention, genetic modification of SSCs using XTNs relates to generating multiple mutations in separate SSCs or SSC lines followed by pooling or combining separate SSCs or SSC lines and injecting into a single recipient male, which relates to generating multiple genetically modified organisms containing one or more mutations is fewer experimental steps and in a shorter timeframe than is possible with other systems. (FIG. 1). The separate SSCs or SSC lines may be fifteen or more.

In some embodiment of the invention, increasing the number of distinct or separate pools or lines of genetically modified SSCs, which may be used to generate a genetically modified organism, does not increase the amount of effort, time, and resources used, as well as does not decrease the efficiency of genetically modified organism production. Multiple separate and distinct genetically modified SSCs may be transplanted into a single sterile recipient. The mixed population of distinct genetically modified SSCs, which are derived from separate SSC pools from two or more pools to fifteen or more pools mature within the sterile recipient. The sterile recipient is then bred with multiple wild type females which may be two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more. These multiple females produce offspring which have incorporated the desired mutation into their germline.

In some embodiment of the invention, increasing the number distinct or separate pools or lines of genetically modified SSCs, which may be used to generate a genetically modified organism, does not increase the amount of effort, time, and resources used, as well as does not decrease the efficiency of genetically modified organism production. The sterile recipient rat may be a recipient for multiple rounds of separate or distinct genetically modified SSCs. The sterile rat may be a recipient of fifteen or more different genetically modified SSCs and breed with twenty or more wild type females to produce fifteen or more separately genetically modified organisms. Following the first round of breeding, the sterile male may be treated to eliminate the first round of genetically modified SSCs and become a recipient of another round of fifteen or more separately or distinct genetically modified SSCs, breed with twenty or more wild type females to produce fifteen or more separate genetically modified organisms. The sterile male may be a recipient of mixed populations of fifteen or more genetically modified SSCs and breed twenty or more wild type females two times or more, three times or more, four times or more, or five times or more.

In some embodiment of the invention, increasing the number distinct or separate pools or lines of genetically modified SSCs, which may be used to generate a genetically modified organism, does not increase the amount of effort, time, and resources used, as well as does not decrease the efficiency of genetically modified organism production. Increasing the number of genetically modified SSCs does not require the effort and resources of other stem cell systems such as embryonic stem (ES) cells or embryos. Increasing the amount of genetically modified ES cells for genetically modified organism production requires an increase in the number of technical steps such as blastocyst injections, as well as the number of oviduct transfer surgeries. In some embodiments of the invention, the method does not comprise blastocyst injection, oviduct transfer, DNA microinjection reimplantation of injected zygotes, or breeding of chimeric progeny. The SSC system may produce fifteen or more separate genetically modified stem cell populations for genetically modified organism production in a single step, while in order to produce fifteen or more separately genetically modified ES cells, fifteen or more separate steps must be performed on all levels of the procedure, which include but are not limited to blastocyst injection, oviduct transfer, zygote production, preparation of DNA, DNA microinjection, reimplantation of injected zygotes or breeding chimeric progeny.

In some embodiment of the invention, genetic modification of SSCs using XTNs relates to generating genetically modified organisms without requiring the steps required in producing genetically modified organisms from alternative stem cells including but not limited to embryonic stem cells, embryo's, induced pluripotent stem (iPS) cells, somatic stem cells. Genetic modification in alternative stem cells includes but is not limited to zygote production, preparation of DNA, DNA microinjection, reimplantation of injected zygotes or breeding chimeric progeny.

In some embodiments, the stem cells of the present invention comprise one or more transposons, one or more inverted tandem repeats (ITRs) of a transposon of variants thereof. In some embodiments, the stem cells of the present invention comprise one or more transposons, one or more inverted tandem repeats (ITRs) of a transposon of variants derived from the sequences of Table 3.

In some embodiments, the present invention comprise one or more transposons, one or more inverted tandem repeats (ITRs) of a transposon wherein the variant sequence inverted tandem repeats are at least 70% homologous to known ITRs and known transposon elements (shown in table 3).

In some embodiments, the present invention comprise one or more transposons, one or more inverted tandem repeats (ITRs) of a transposon wherein the variant sequence inverted tandem repeats are at least 75% homologous to known ITRs and known transposon elements (shown in table 3).

In some embodiments, the present invention comprise one or more transposons, one or more inverted tandem repeats (ITRs) of a transposon wherein the variant sequence inverted tandem repeats are at least 80% homologous to known ITRs and known transposon elements (shown in table 3).

In some embodiments, the present invention comprise one or more transposons, one or more inverted tandem repeats (ITRs) of a transposon wherein the variant sequence inverted tandem repeats are at least 85% homologous to known ITRs and known transposon elements (shown in table 3).

In some embodiments, the present invention comprise one or more transposons, one or more inverted tandem repeats (ITRs) of a transposon wherein the variant sequence inverted tandem repeats are at least 90% homologous to known ITRs and known transposon elements (shown in table 3).

In some embodiments, the present invention comprise one or more transposons, one or more inverted tandem repeats (ITRs) of a transposon wherein the variant sequence inverted tandem repeats are at least 95% homologous to known ITRs and known transposon elements (shown in table 3).

In some embodiments, the present invention comprise one or more transposons, one or more inverted tandem repeats (ITRs) of a transposon wherein the variant sequence inverted tandem repeats are at least 96% homologous to known ITRs and known transposon elements (shown in table 3).

In some embodiments, the present invention comprise one or more transposons, one or more inverted tandem repeats (ITRs) of a transposon wherein the variant sequence inverted tandem repeats are at least 97% homologous to known ITRs and known transposon elements (shown in table 3).

In some embodiments, the present invention comprise one or more transposons, one or more inverted tandem repeats (ITRs) of a transposon wherein the variant sequence inverted tandem repeats are at least 98% homologous to known ITRs and known transposon elements (shown in table 3).

In some embodiments, the present invention comprise one or more transposons, one or more inverted tandem repeats (ITRs) of a transposon wherein the variant sequence inverted tandem repeats are at least 99% homologous to known ITRs and known transposon elements (shown in table 3).

Generating undifferentiated SSCs requires using SSC media and feeder media using DMEM-high glucose+Sodium Bicarbonate Medium contains Dulbecco's Modified Eagle's Medium-high glucose (Sigma, D5648); 1.5 g Sodium Bicarbonate (Sigma, S5761), 1 L sterile water which are filtered using a 0.2 um filter unit and stored at 4 C; SSC Feeder Medium contains 225 mL DMEM-high glucose+sodium bicarbonate; 25 mL Heat Inactivated Fetal Bovine Serum: FBS (Tissue Culture Biologicals, 104), which are filtered using a 0.2 um filter unit and stored at 4 C; 0.1% Gelatin is generated by dissolving 1 g gelatin from Porcine Skin-Type A (Sigma, G1890) in 1 L ultrapure water. Gelatin is autoclaved on liquid cycle and stored at 4 C; Recombinant Rat GDNF (rR-GDNF; R&D Systems, 512-GF-010) is supplied at 10 ug, then reconstituted to 100 ug/mL (100 ng/uL) by adding 100 uL 1×PBS/0.1% BSA (0.001 g BSA-Calbiochem 126609 in 10 mL Sigma D8537 1×PBS-sterile filtered). rR-GDNF is pipetted up and down to mix, but not vortexed. Do not freeze thaw rR-GDNF more than 2× and store at −20 C, Recombinant Fibroblast Growth Factor-Basic Human (rbH-FGF; Sigma, F0291) is supplied at 25 ug, then reconstituted to 25 ug/mL (25 ng/uL) by adding 1 mL-1×PBS/0.1% BSA (0.001 g BSA-Calbiochem 126609 in 10 mL Sigma D8537 1×PBS-sterile filtered). rbH-FGF is pipetted up and down to mix but not vortexed. Do not freeze thaw rbH-FGF more than 2× and store at −20 C, Dilute 2-Mercaptoethanol (Sigma M3148) is prepared by adding 4.7 uL stock to 6 mL DHF12 (Sigma D8437).

Spermatogonial Culture Medium (SG Medium) is made by preparing reagents such as in Table 1. in the SG medium the rR-GDNF final concentration is 20 ng/ml, rbH-FGF 20 ng/ml, 2-mercaptoethanol 100 μM, L-glutamine 4 mM final concentration—media's overall final concentration glutamine in 6 mM, B27 Supplement minus vitamin A, 1×. Sterile filter the medium using 0.2 um filter unit, and store at 4 C. Media over two weeks old is not to be used.

Subculturing and preserving rat SSCs for propagation and archiving requires preparing fibroblasts feeder cell lines and cryopreservation of SSCs.

In order to prepare fibroblasts feeder cell lines coat dish with 0.1% gelatin and incubate at 37 C -1 hour and wash 1× with 1×PBS. Thaw IRR mouse embryonic fibroblasts by placing frozen vial at 37 C immediately after removing from liquid nitrogen until ice crystals disappear. Transfer contents into 9 mL of 37 C DR4 Feeder medium. Spin at 1000 rpm for 5 minutes, discard supernatant, and resuspend in SSC Feeder medium. Plate on gelatin coated surface in SSC Feeder medium for 16-48 hr. Using 6-well plate—0.43×10̂6 cells/well and 10 cm dish—2.6×10̂6 cells/dish rinse with lx PBS and then pre-incubate in SG medium for an additional 16-48 hr. The SG medium used for pre-incubation is then discarded and spermatogonia are passaged onto the MEFs in fresh SG medium (Table 1).

In order to sub-culture SSC lines The initial passage of spermatogonial cultures after thawing onto MEF feeder layers requires a 1:1 to 1:2 split into the same size wells at 10-12 days after their initial seeding onto the MEFs. Additionally, if required, fresh MEFs (2×104/cm2) can also “spiked” into the on-going spermatogonial cultures on day 11-12 so to by-pass the need to immediately passage the spermatogonia before expanding to larger numbers. Using 6-well plates—0.19×10̂6 cells/well and 10 cm dish—1.16×10̂6 cells/dish once established after the first couple passages on MEFs post-thaw, cultures of spermatogonia are passaged at ˜1:3 dilutions onto a fresh monolayer of MEFs every 10-14 days at ˜3×104 cells/cm2 for over 5 months (i.e. ˜12 passages).

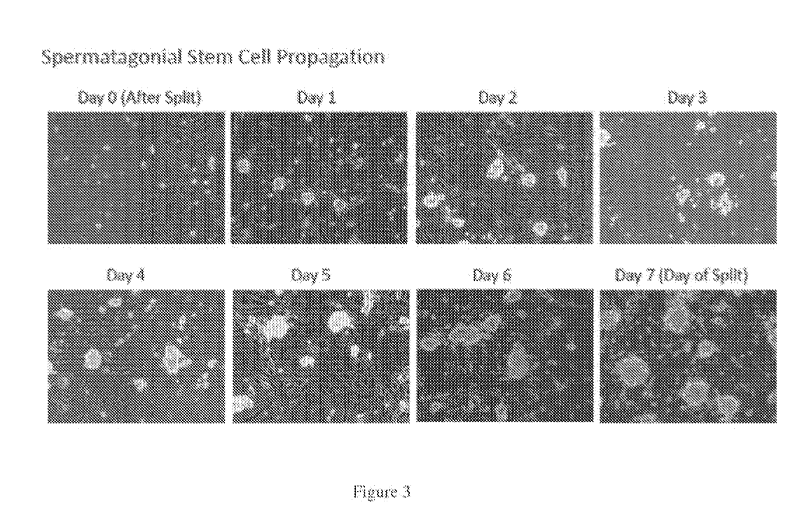

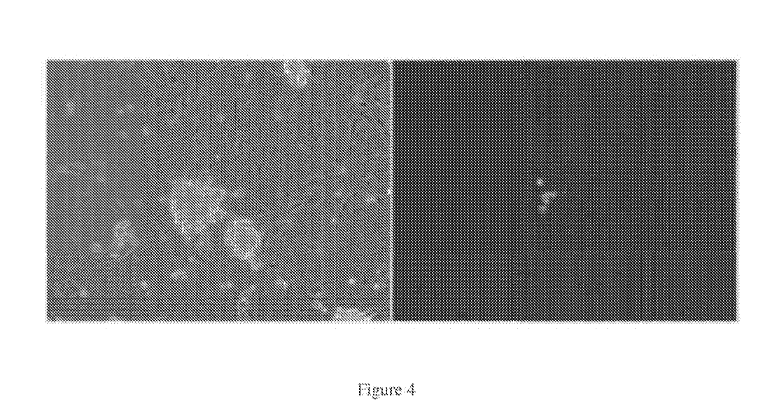

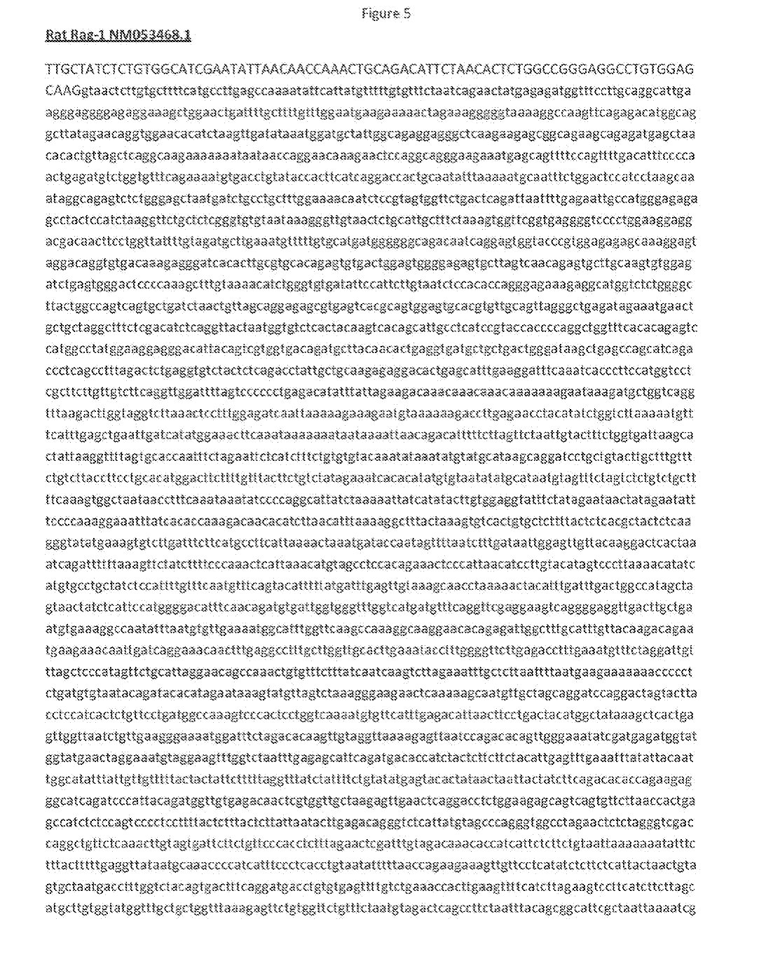

Requirements for passaging and propagation are shown in Table 2. For passaging, cultures are first harvested by gently pipetting them free from the MEFs. After harvesting, the “clusters” of spermatogonia are dissociated by gentle trituration with 20-30 strokes through a P1000 pipet tip in SG Medium. The dissociated cells are pelleted at 400×g for 4 minutes and the number of cells recovered during each passage is determined by counting on a Hemocytometer (Note: spermatogonial clusters are not disrupted for counting until the second passage on MEFs). Spermatogonia are easily distinguished during counting as the predominant population of smaller, round cells with smooth surfaces, as compared to occasionally observed, larger and often irregular shaped irradiated MEFs. Typically, 2-4×106 spermatogonia can be harvested from a single, 10 cm dish (FIG. 3).

Cryopreservation of SSCs for archiving is achieved by preparing Spermatogonial Freezing Medium (SG Freezing Medium) by adding DMSO at a concentration of 10% (v/v) in SG Medium. Filter-sterilize and cool the prepared freezing medium on ice prior to use. Prepare a 5100 Cryo 1° C. Freezing Container “Mr. Frosty” (Thermo Fisher Scientific Nalgene, Inc.,15-350-50) by adding 200 ml fresh isopropanol to the outer chamber. Chill the container by equilibrating it to ˜4 C in a refrigerator prior to use. Suspend the harvested spermatogonial pellet in ice-cold, SG Freezing Medium at 2×105 to 2×106 cells/ml and then aliquot stocks into cryovials (Thermo Fisher Scientific Nalgene, 03-337-7D) at 1 ml/vial. Work quickly and place filled cryovials on ice while finishing aliquots. Place cryovials of spermatogonial stocks into the pre-chilled “Mr Frosty” and close container firmly. Store the freezing container of spermatogonial stocks at −80 C for 24 hours, then transfer vials into a liquid nitrogen cryostorage unit.

Transfection of Rat Spermatogonia with Xanthomonas TAL Nucleases (XTNs) constructs and plasmid DNA, such as selection using fluorescent markers and homologous recombination vectors, using Lipofectamine 2000 requires a number of reagents and methods.

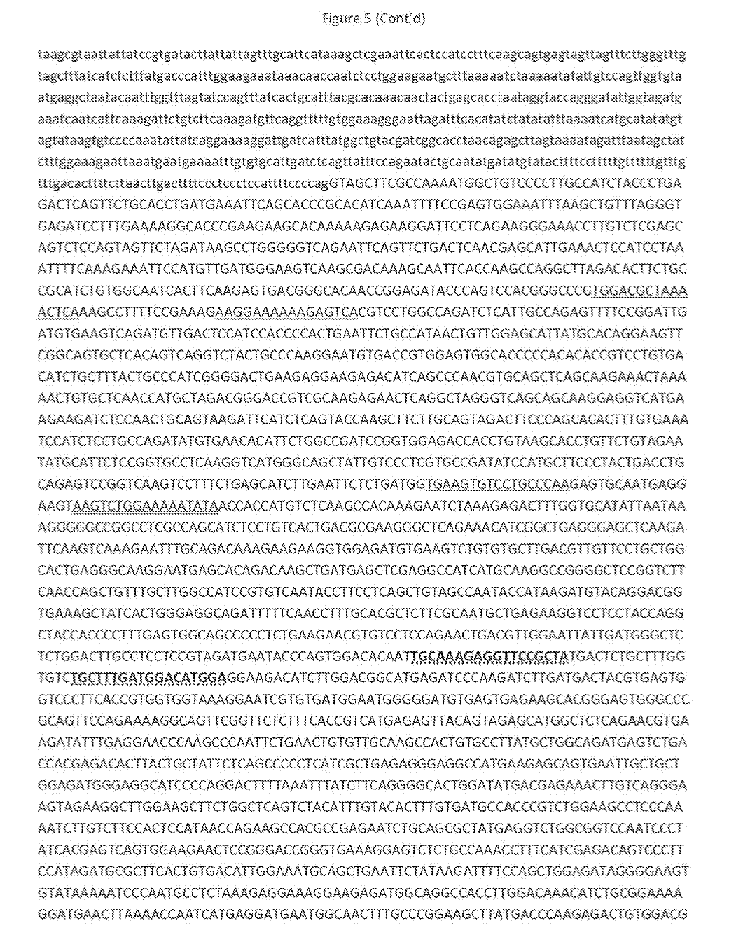

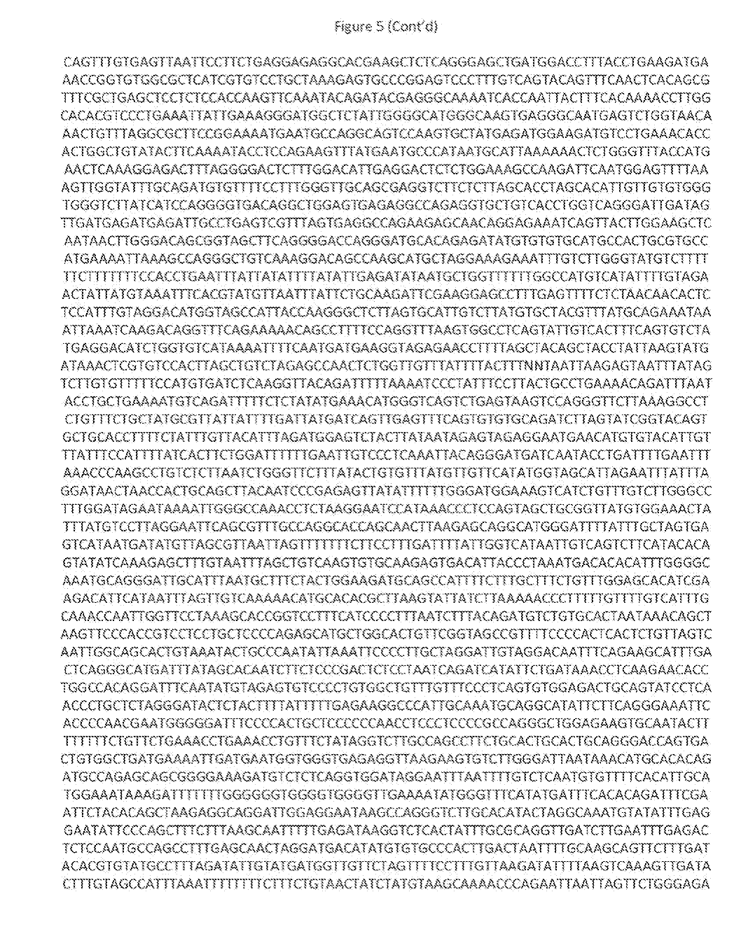

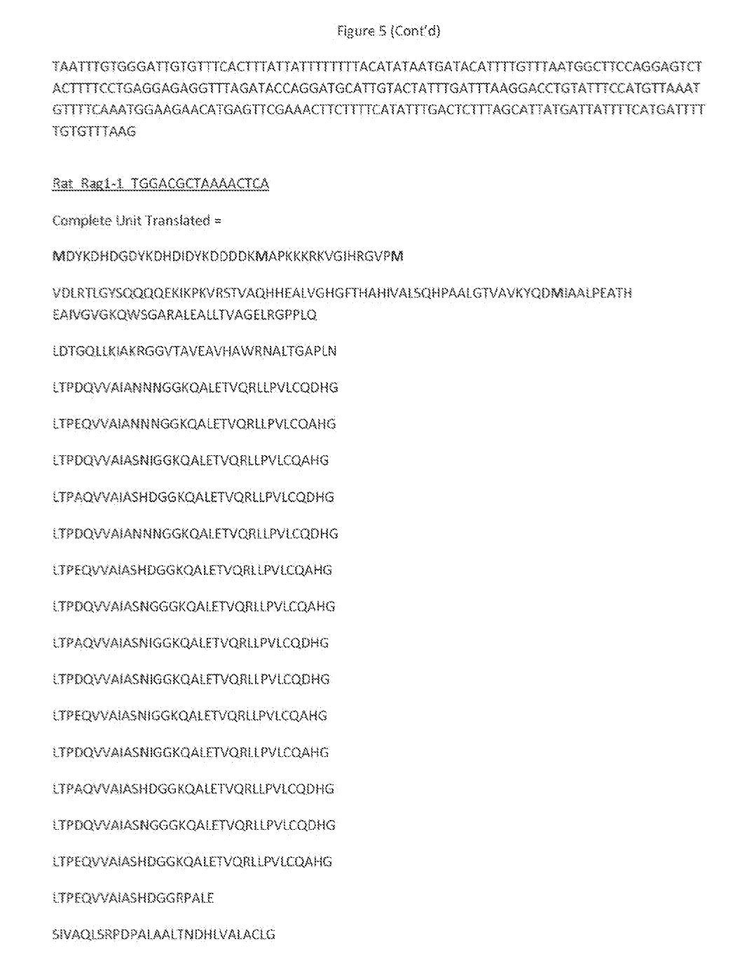

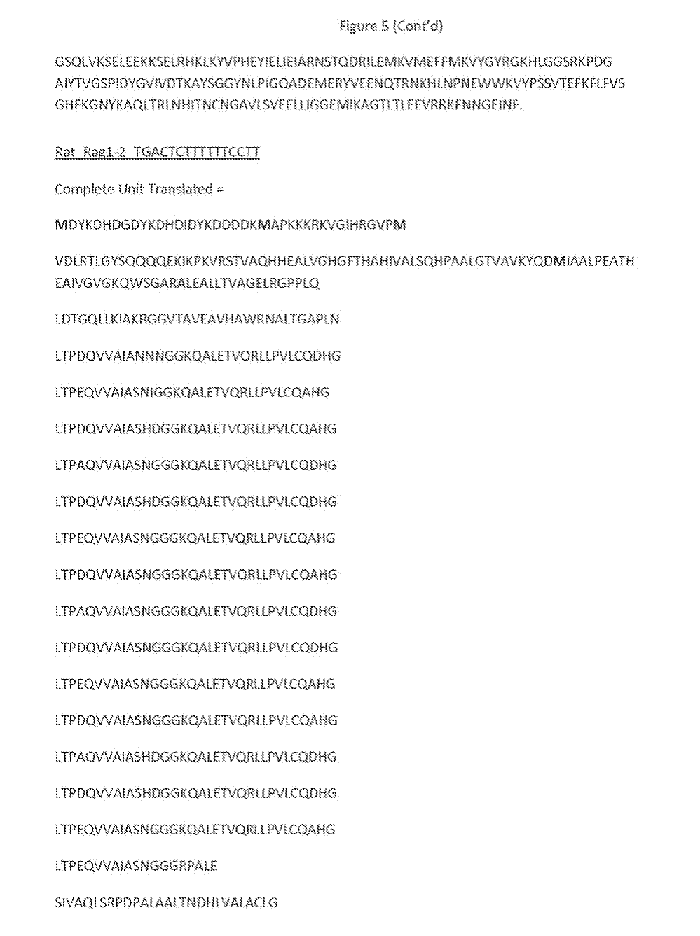

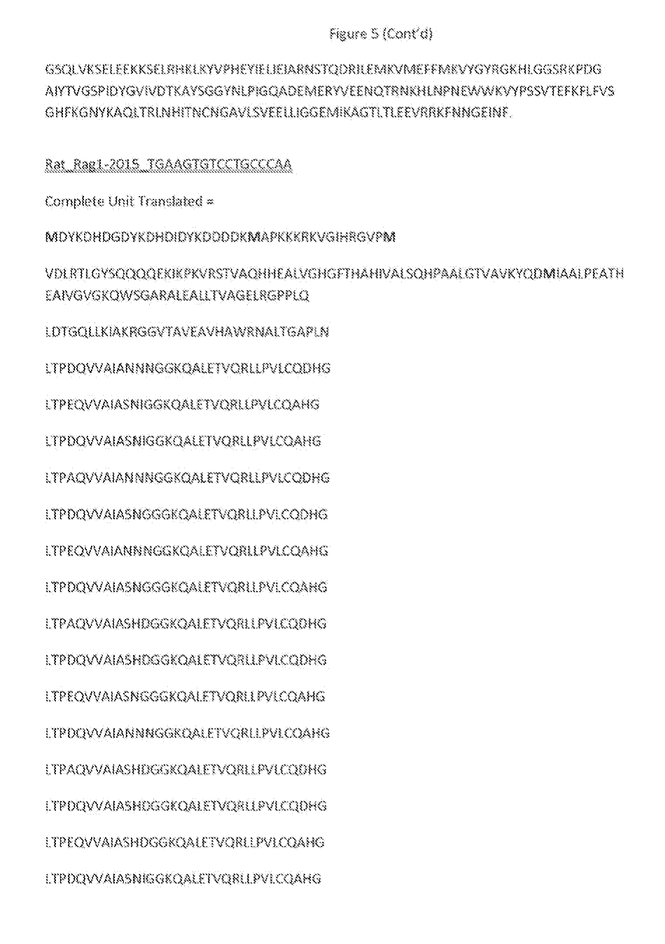

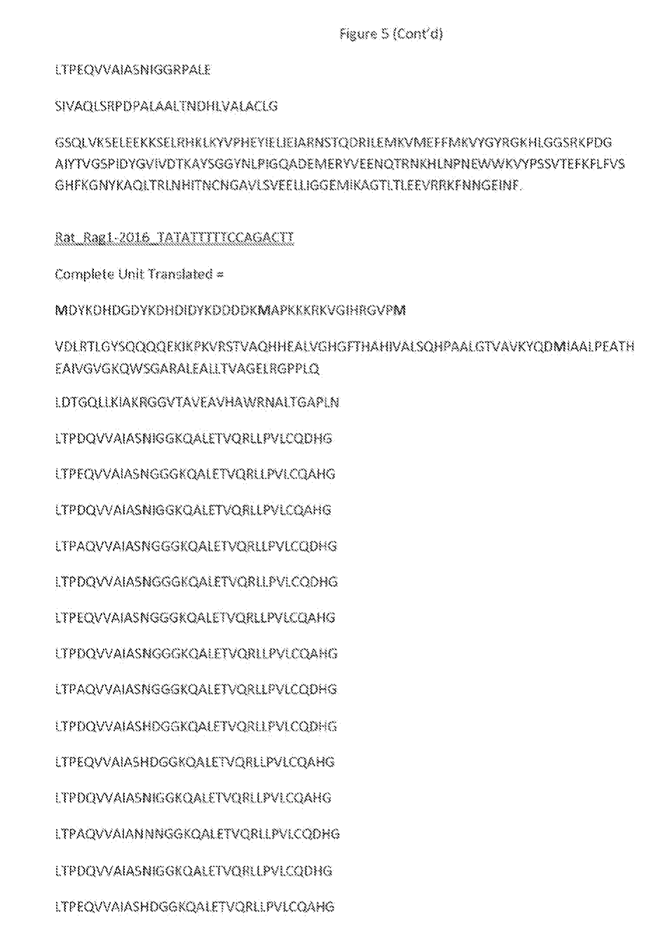

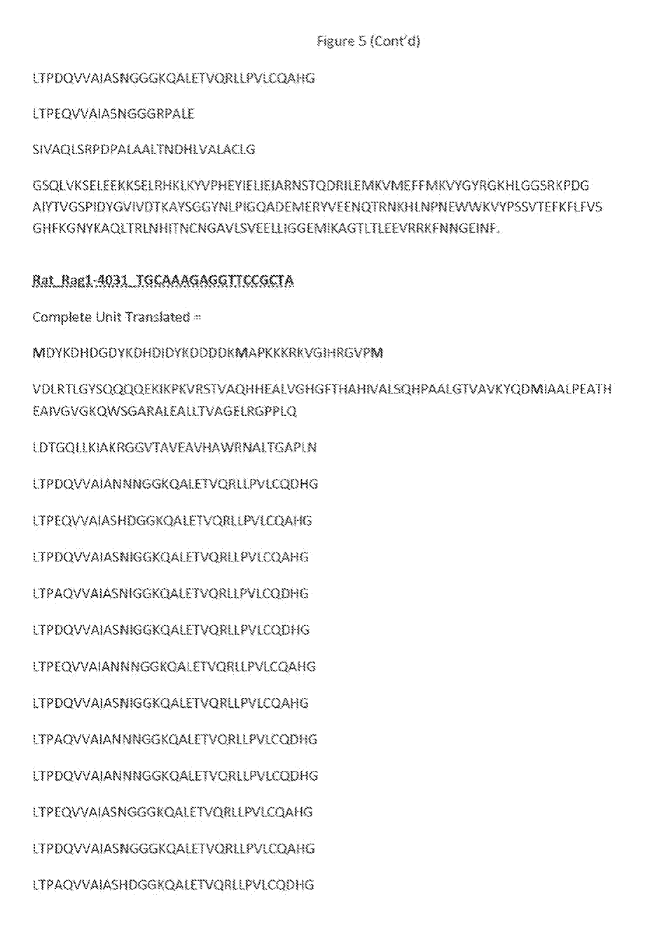

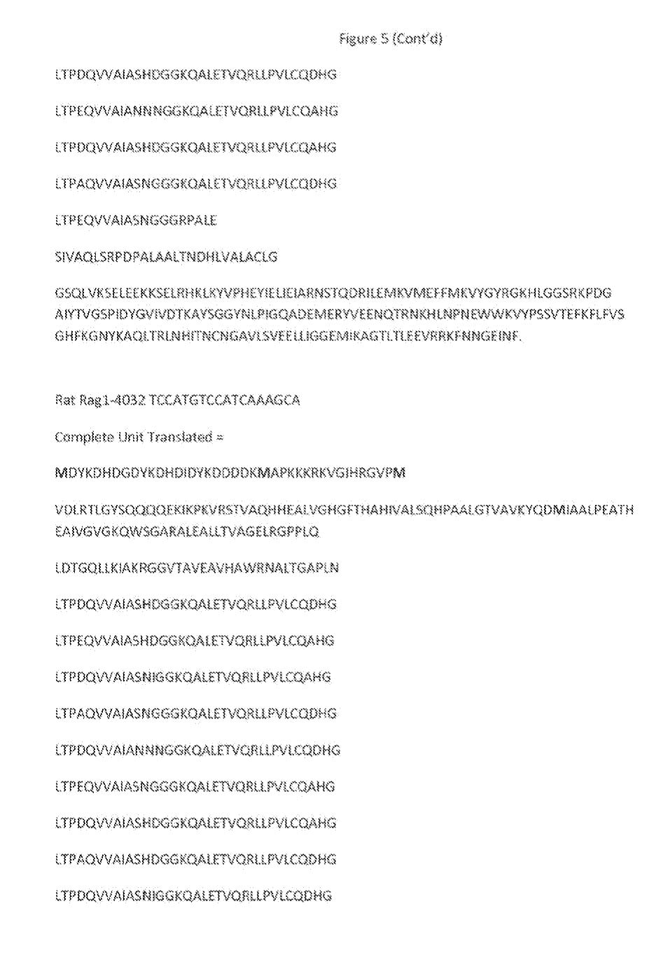

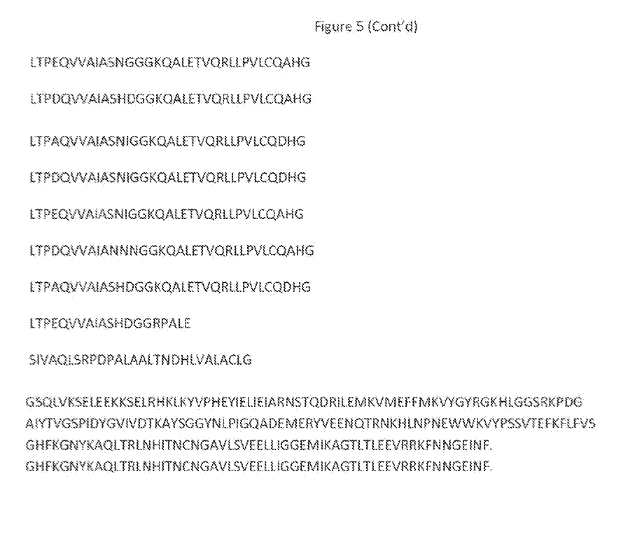

XTN constructs and plasmids used for transfection into SSCs typically contain XTN encoding gene which targets specific sites within the genome of an organism (FIG. 5).

Reagents include undifferentiated spermatogonia, SG Medium (pre-warmed), Opti-MEM (cat. no. 31985-062; Invitrogen, Inc.), Lipofectamine 2000 (cat. no. 11668-019; Invitrogen), highly purified XTN construct and plasmid DNA containing selection markers or homologous recombination vectors in TE buffer at 1-2 μg/μl, Gelatin-coated plates, and plates with fresh MEF feeder layers.

Prepare a Transfection Mixture containing Lipofectamine 2000 (Invitrogen) XTN construct and plasmid DNA in Opti-MEM, as follows: In a 1.5 ml microfuge tube, dilute 1 μg DNA/100 μl Opti-MEM. In a separate 1.5 ml microfuge tube, dilute 2 μl Lipofectamine 2000/100 μl Opti-MEM. Incubate tubes separately for 5-10 min. Combine contents of each tube together and incubate at room temperature for at least 20 minutes (but no longer than 6 hr) to obtain the Transfection Mixture. During this incubation step, proceed to harvesting cells for transfection. Harvest cultures of proliferating spermatogonia grown on MEFs. If using proliferating cultures of spermatogonia maintained on MEF feeder layers, first plate the cells onto a fresh gelatin-coated plate and incubate for 30-45 min (37° C., 5% CO2) to deplete the number of residual MEFs present in the cell suspension. Suspend spermatogonia to ˜106 cells/ml in SG Medium, or DHF12-FBS+30 μM 2ME. Add Transfection Mixture to the cell suspension at a ratio of 20% volume Transfection Mixture: 80% volume spermatogonial suspension, and incubate at 37° C., 5% CO2 for 40-120 min (routinely 80 min) in a vented tube. As a typical example, 40 μl volume of the Transfection Mixture is used to transfect ˜2×105 spermatogonia in a total transfection volume of 200 μl. During transfections lasting longer than 1 h, mix the transfection by gently pipetting cells up and down two times midway through the incubation period. After the transfection incubation period, wash spermatogonia by first suspending the transfection suspension to 20 times its volume using fresh culture medium (i.e. 4 ml medium/200 μl transfection reaction), and then pellet the cells for 5 min at 400×g. Discard the supernatant fluid, and wash the pellet(s) two additional times using fresh culture medium at an equivalent of the 20× volume/wash. After the third wash, suspend the cell pellet in fresh medium and then plate transfected cells onto fresh MEF feeder layers for selection of genetically modified spermatogonial lines.

Clonal selection for genetically modified SSCs is done by using the following reagents: established, proliferating line of rat spermatogonial stem cells, geneticin selective antibiotic: G418 (cat no 11811-031, Invitrogen Inc.), DNA Constructs expressing a resistance gene that selects for survival in G418 containing medium (i.e. neomycin phosphotransferase gene), fibroblast feeder cell line expressing a resistance gene that selects for survival in G418 containing medium.

After transfecting spermatogonia from an established proliferating line with XTN construct and plasmid DNA or selectable marker, particles, the treated spermatogonia are plated directly into SG Medium at an equivalent of ˜3×105 spermatogonia/well (9.5 cm2) in a 6-well plate containing freshly prepared MEFs. The transfected spermatogonia are then allowed to proliferate in cell number for ˜18 days after transfection with plasmid DNA. The culture medium is replenished every two days; and, fresh MEFs are spiked onto cultures of the transfected spermatogonia after ˜10 days. At ˜18 days following gene-transfer with XTN construct and plasmid DNA or selectable marker, cultures are harvested and then passaged onto freshly prepared MEFs in SG medium and maintained for an additional 2-3 days before initiating clonal selection in SG medium containing ˜75 μg/ml G418 (Invitrogen, Inc.). After initiating selection, cultures are fed fresh SG medium containing G418 every two days during an 8-10 day selection period. Thereafter, cells are fed every two days using SG medium alone to expand clonally enriched lines of rat spermatogonia that can be used to produce transgenic rats, as described in the following sections.

In some embodiment of the invention, genetic modification of SSCs produced using XTNs relates to generating multiple mutations in the same SSC or SSC line, which relates to generating genetically modified organisms with multiple mutations. The embodiment relates to transfection with multiple XTN constructs targeting multiple DNA sequences and locations within the same SSC or SSC line in a single transfection. The embodiment of the invention relates to clonal selection and screening for multiple mutations in single SSCs or SSC lines.

In some embodiment of the invention, genetic modification of SSCs produced using XTNs relates to generating multiple mutations in the same SSC or SSC line in multiple and consecutive experiments or transfections. The embodiment of the invention relates to clonal selection and screening for multiple mutations in single SSCs or SSC lines. The embodiment of the invention relates to generating genetically modified organisms or a colony of genetically modified organisms with multiple mutations in single SSCs or SSC lines.

In some embodiment of the invention, genetic modification of SSCs produced using XTNs relates to generating multiple mutations in separate SSCs or SSC lines followed by pooling or combining separate SSCs or SSC lines and injecting into a single recipient male, which relates to generating multiple genetically modified organisms containing one or more mutations. In the embodiment of the invention SSCs or SSC lines are separated by including, but not limited to, different media, colonies or transfection dishes. The separated SSCs or SSC lines undergo one or more experiments or transfections. The separate SSCs or SSC lines are then brought together for production of multiple genetically modified organisms in a single injection into a recipient male followed by a single breeding step.

In some embodiment of the invention, the SSCs are derived from an organism. The SSCs may be collected by spermatocyte harvest, the SSCs may be selected and purified using laminin selection, and propagated, cryopreserved and validated by cell surface marker identification.

In some embodiment of the invention, the SSCs are derived from a tissue sample. The SSCs may be collected by spermatocyte harvest, the SSCs may be selected and purified using laminin selection, and propagated, cryopreserved and validated by cell surface marker identification.

In some embodiment of the invention, the SSCs are derived from cells. The SSCs may be collected by spermatocyte harvest, the SSCs may be selected and purified using laminin selection, and propagated, cryopreserved and validated by cell surface marker identification.

In some embodiment, the SSCs used for production of organisms are derived from an organism or tissue with a well-characterized disease state. The SSCs are used for the production of organisms, which may be further genetically modified.

In some embodiment, the SSCs used for production of organisms are derived from a well-characterized disease state wherein the disease state is a metabolic disorder, which is not limited to diabetes. The SSCs are used for the production of organisms which may be further genetically modified.

In some embodiment, the SSCs used for production of organisms are derived from a well-characterized disease state wherein the disease state is an oncology disorder, which is not limited to prostate cancer. The SSCs are used for the production of organisms which may be further genetically modified.

In some embodiment, the SSCs used for production of organisms are derived from a well-characterized disease state wherein the disease state is an autoimmune disorder, which is not limited to arthritis. The SSCs are used for the production of organisms which may be further genetically modified.

In some embodiment, the SSCs used for production of organisms are derived from a well-characterized disease state wherein the disease state is a cardiovascular disorder, which is not limited to atherosclerosis. The SSCs are used for the production of organisms which may be further genetically modified.

In some embodiment, the SSCs used for production of organisms are derived from a well-characterized disease state wherein the disease state is a neurodegenerative disorder, which is not limited to Alzheimer's disease. The SSCs are used for the production of organisms which may be further genetically modified.

In some embodiment, the SSCs used for production of organisms are derived from a well-characterized disease state wherein the disease state is a behavioral disorder, which is not limited to Schizophrenia. The SSCs are used for the production of organisms which may be further genetically modified.

In some embodiment, the SSCs used for production of organisms are derived from induced pluripotent stem (iPS) cells from a well-characterized disease state wherein the disease state is a metabolic disorder, which is not limited to diabetes. The SSCs are used for the production of organisms which may be further genetically modified.

In some embodiment, the SSCs used for production of organisms are derived from induced pluripotent stem (iPS) cells from a well-characterized disease state wherein the disease state is an oncology disorder, which is not limited to prostate cancer. The SSCs are used for the production of organisms which may be further genetically modified.

In some embodiment, the SSCs used for production of organisms are derived from induced pluripotent stem (iPS) cells from a well-characterized disease state wherein the disease state is an autoimmune disorder, which is not limited to arthritis. The SSCs are used for the production of organisms which may be further genetically modified.

In some embodiment, the SSCs used for production of organisms are derived from induced pluripotent stem (iPS) cells from a well-characterized disease state wherein the disease state is a cardiovascular disorder, which is not limited to atherosclerosis. The SSCs are used for the production of organisms which may be further genetically modified.

In some embodiment, the SSCs used for production of organisms are derived from induced pluripotent stem (iPS) cells from a well-characterized disease state wherein the disease state is a neurodegenerative disorder, which is not limited to Alzheimer's disease. The SSCs are used for the production of organisms which may be further genetically modified.

In some embodiment, the SSCs used for production of organisms are derived from induced pluripotent stem (iPS) cells from a well-characterized disease state wherein the disease state is a behavioral disorder—which is not limited to Schizophrenia. The SSCs are used for the production of organisms which may be further genetically modified.

In some embodiment, the SSCs used for production of organisms are derived from induced pluripotent stem (iPS) cells from a well-characterized genetic background.

In some embodiment, the SSCs used for production of organisms are derived from induced pluripotent stem (iPS) cells from a well-characterized genetic background wherein the genetic background is associated with different established strains of organism.

In some embodiment, the SSCs used for production of organisms are derived from induced pluripotent stem (iPS) cells from a well-characterized genetic background wherein the genetic background is associated with known ethnic or regional genetic make-ups.

In one embodiment, SSCs containing site-specific mutations are generated to produce genetically modified organisms.

In another embodiment, SSCs containing site specific mutations are generated to produce genetically modified mammals.

In another embodiment, SSCs containing site-specific mutations are generated to produce genetically modified rodents.

In another embodiment, SSCs containing site-specific mutations are generated to produce genetically modified rats.

In another embodiment, SSCs containing site-specific mutations are generated to produce genetically modified mice.

In another embodiment, SSCs containing site-specific mutations are generated to produce genetically modified pigs

In another embodiment, SSCs containing site-specific mutations are generated to produce genetically modified rabbits

In another embodiment, SSCs containing site-specific mutations are generated to produce genetically modified guinea pigs.

In another embodiment, SSCs containing site-specific mutations are generated to produce genetically modified dogs.

In another embodiment, SSCs containing site-specific mutations are generated to produce genetically modified cats.

In another embodiment, SSCs containing site-specific mutations are generated to produce genetically modified goats.

In another embodiment, SSCs containing site-specific mutations are generated to produce genetically modified chickens.

In another embodiment, SSCs containing site-specific mutations are generated to produce genetically modified non-human primates.

In another embodiment, SSCs containing site-specific mutations are generated to produce genetically modified ferrets.

In another embodiment, SSCs containing site-specific mutations are generated to produce genetically modified birds.

In another embodiment, SSCs containing site-specific mutations are generated to produce genetically modified farm animals.

In another embodiment, SSCs containing site-specific mutations are generated to produce genetically modified fish.

In another embodiment, SSCs containing site-specific mutations are generated to produce genetically modified slamonoids.

In another embodiment, SSCs containing site-specific mutations are generated to produce genetically modified carp.

In another embodiment, SSCs containing site-specific mutations are generated to produce genetically modified tilapia.

In another embodiment, SSCs containing site-specific mutations are generated to produce genetically modified tuna.

In another embodiment, the invention provides kits that are used to produce site specific-mutations in stem cells, which can be used to generate genetically modified organisms. The kits typically include one or more site-specific genetic engineering technology, such as XTNs. The kit may also contain one or more sets of stem cells for site-specific modification. The stem cells may include, but is not limited to spermatogonial stem cells (SSCs), as well as media and conditions necessary for growing SSCs. The kits may include exogenous sequences for site-specific genomic introduction, such as but not limited to reporter genes or selectable markers. The kits may include instructions for (i) introducing the XTNs into the stem cells (ii) identifying stem cells which have been site specifically modified (iii) growing site-specifically modified stem cells in media or conditions necessary and to numbers required for stem cells to produce genetically modified organisms (iv) using the grown stem cells to produce a genetically modified organism (v) identifying which organisms or progeny harbor the site-specific mutation of interest.

In some embodiment, the invention provides a kit which includes a mixed population of different or distinct genetically modified SSCs which may be custom made. The mixed population of genetically modified SSCs may be provided in suitable quantities for direct injection into a sterile male recipient for the production of multiple genetically modified organisms in a single step. The mixed population of separate or distinct genetically modified SSCs may consist of at least two genetically modified SSCs, at least two genetically modified SSCs, at least three genetically modified SSCs, at least four genetically modified SSCs, at least five genetically modified SSCs, at least six genetically modified SSCs, at least seven genetically modified SSCs, at least eight genetically modified SSCs, at least nine genetically modified SSCs, at least ten genetically modified SSCs, at least twenty genetically modified SSCs, at least thirty genetically modified SSCs, at least forty genetically modified SSCs, at least fifty genetically modified SSCs, at least one hundred genetically modified SSCs, at least one thousand genetically modified SSCs, at least ten thousand genetically modified SSCs, at least thirty thousand genetically modified SSCs or with genetically modified SSCs which harbor genetic modification within every gene in the organisms genome.

In some embodiment, the invention provides a kit which includes one or more sets of SSCs for site-specific modification. The sets of SSCs may be derived from well-characterized organisms having different disease states. The SSCs may contain multiple mutations, which may be derived from genetic modification or naturally or by any method. The kit may include the media and conditions to grow disease state SSCs, as well as the sterile recipient male for the production of genetically modified organisms.

In some embodiment, the invention provides a kit which includes the necessary tools for the derivation of SSC lines from an organism or tissue sample, as well as the necessary tools to genetically modify the derived SSC and produce a genetically modified organism from the derived SSCs. The kit may include cell collection tools such as spermatocytes for harvest, and SSC selection tools such as laminin selection, and SSC propagation and cryopreservation tools as well as SSC validation tools which may include cell surface marker staining The kit may also include media and conditions for growing the SSCs, tools for genetic modification of the SSCs as well as sterile recipient males for production of genetically modified organisms from the SSCs.

In some embodiment, the invention provides a kit, which includes SSCs which have been generated from induced pluripotent stem (iPS) cells. The iPS cells may be derived from well characterized different genetic backgrounds including disease states as well as regional, strain, ethnic genetic backgrounds. The kit may also include media and conditions for growing the SSCs, tools for genetic modification of the SSCs as well as sterile recipient males for production of genetically modified organisms from the SSCs.

Germline transmission from genetically modified SSCs can be carried out by using the following reagents: Disposable Pasteur Pipettes (cat. no. 13-678-20C, Thermo Fisher Scientific Inc.), 30G Precision Glide Needles (cat. no. 305106, BD, Inc.), 1 ml Syringes (cat. no. 309602, BD Inc.), Busulfan (cat. no. 154906, MP Biomedicals), Dimethyl Sulfoxide (DMSO) (cat. no. 317275, Calbiochem), Trypan Blue (cat. no. T6146-25G, Sigma Inc.), Triadine Prep Solution, (10% povidone iodine solution, cat. no. 10-8208, Triad Disposables), Ethanol 200 Proof (cat. no. 111000200, Pharmco-AAPER), PBS: Dulbecco's phosphate-buffered saline (PBS; cat. no. D8537, Sigma Inc.) 200 mg/L KCl (w/v), 200 mg/L KH2PO4 (w/v), 8 g/L NaCl (w/v), 1.15 g/L Na2HPO4 (w/v)., Kimwipes (cat. no. 34155, Kimberly-Clark), Bead Sterilizer; Germinator 500 (Cellpoint Scientific Inc), Flaming/Brown Micropipette Puller; Model P-97 (Sutter Instruments Co.),Glass Capillaries for needles; 100 μl micropipette (cat. no 1-000-1000, Drummond Scientific Co.), Heat Therapy Pump (cat. no HTP-1500, Kent Scientific Corporation or other suitable model), Reusable Warming Pad (cat. no. TPZ-1215EA, Kent Scientific Corporation), 10 ml Syringes (cat. no. 309604, BD, Inc.), Acepromazine (cat. no. 038ZJ03, Vedco), Rompun (cat. no. LA33806A, Lloyd Laboratories) Ketaset (cat. no. 440761, Fort Dodge Animal Health), Buprenex Injectable (cat. no. 12496-0757-1, Reckitt Benckiser), Shaving Razors—Stainless Steel Surgical Prep Blades (cat. no. 74-0001, Personna), Suture Thread; Spool Suture (cat. no. SUT-15-2, Roboz Surgical Inc.), Suture Needles; Eye 3/8 circle (cat. no. RS-7981-4, Roboz Surgical Inc.), Michel Wound Clips (cat. no. RS-9272, Roboz Surgical Inc.), Michel Wound Clip Forceps (cat. no. RS-9294, Roboz Surgical Inc.), Ear Puncher—2 mm diameter (cat. no. RS-9902, Roboz Surgical Inc.), Hemostat (cat. no. RS-7110, Roboz Surgical Inc.), Straight Sharp Microdissecting Scissors (cat. no. RS-5882, Roboz Surgical Inc.) Curved, Sharp Microdissecting Scissors (cat. no. RS-5883, Roboz Surgical Inc.), Full-Serve Microdissecting Forceps (cat. no. RS-5137, Roboz Surgical Inc.), Straight Tip, Dumostar Tweezers (cat. no. RS-4978, Roboz Surgical Inc.), 5/45 INOX Tweezers (cat. no. RS-5005, Roboz Surgical Inc.) Polyethylene capillary tubing (cat. No. 19-0040-01, GE Healthcare, Inc.), 24 day old, busulfan-treated, male, Sprague Dawley rats.

Generation of recipient-founders by testicular transplantation is carried out by using busulfan-treated wildtype Sprague Dawley rats (Harlan, Inc), or male-sterile DAZL-deficient, Sprague Dawley rats at 24 days of age can be used as recipients for spermatogonial lines. To prepare recipients for transplanting spermatogonia, rats arrive from the supplier at 8-10 days of age, together with mother, 14-16 days prior to the transplantation procedure which is performed at 24 days of age. At 12 days of age (i.e. 12 days prior to the transplantation procedure), each rat is administered a single dose of busulfan (12.5 mg/kg, i.p. for wildtype Sprague Dawley rats; 12.0 mg/kg for DAZL-deficient Sprague Dawley rats), and then housed in a quiet, clean and well ventilated location within an approved animal facility. Under guidelines of an approved safety plan*, a 4 mg/ml working stock of busulfan in 50% DMSO is prepared by first dissolving busulfan in 100% DMSO at 8 mg/ml, and then adding and equal volume of filter-sterilized, deionized water.

On the day of transplantation, genetically modified rat spermatogonia are harvested from culture and suspended in ice cold, culture medium (i.e. either SG medium or DHF12-FBS-2ME) at concentrations ranging from 4-6×105 spermatogonia/100 pl. The cellular suspension is transferred to a sterile microfuge tube and maintained on ice until the time of transplantation. Just prior to transplantation, the cell suspension is supplemented with a 20% volume of a filter-sterilized, 0.04% trypan blue solution made fresh in PBS the same day. Once spermatogonia are harvested, the first busulfan-treated recipient rat is anesthetized by intraperitoneal (i.p.) injection of a cocktail containing 100 mg/ml ketaset, 20 mg/ml rompun, and 10 mg/ml acepromazine at 0.1 ml/100 g body weight to achieve a surgical plane of anesthesia (as demonstrated by the lack of a pedal reflex in the toe pinch test). The recipient is layed on it's back. The abdominal skin is then opened just rostral to the pelvis, and the testis is exposed. The efferent ductules leading into the rete testis are then accessed by blunt dissection using micro-dissection forceps. The ductules are further dissected up to the base of their respective testis to yield visible access to the rete, which will be the site of injection. Once the rete is exposed, the harvested spermatogonial suspension is mixed gently by pipetting up and down ˜5 times with a p200 tip and then ˜70-80 μl of the suspension is loaded into a 100 μl glass capillary injection needle (˜50 μm opening) using a flame pulled, transfer pipette (i.e. made from Pasteur pipettes) and rubber squeeze bulb. The injection needle containing spermatogonia is manually inserted into the rete of the testis, and the cells are transferred into the testis by injection using a stationary 10 ml syringe (i.e. simply taped to the work bench), which is connected to the glass capillary injection needle by flexible plastic tubing. The injected testis is then carefully placed back into the abdominal cavity and the same procedure can be performed on the contra-lateral testis to achieve more optimal breeding. Once injected and placed back in the abdominal cavity, the abdominal wall (sutured) and skin (wound clips) are surgically closed. The procedure can then be repeated on subsequent recipients using the same spermatogonial suspension. Spermatogonial suspensions can be maintained on ice in SG medium for up to 5 hours during the transplantation of multiple recipients.

After surgically closing the abdominal cavity and skin, all animals are maintained on a warming pad set to 34 oC and receive post-operative care to assure their safe recovery from anesthesia and to alleviate pain and distress. For recovery from anesthesia, each animal is observed with respect to its breathing rate, muscle control and external stimuli until ambulatory, prior to being housed in a quiet, well ventilated location within the animal facility.

As a post-operative analgesic to alleviate pain, each rat is administered a single dose of buprenorphine hydrochloride (25 μg/kg) (Buprenex Injectable, Reckitt Benckiser) as it starts to regain consciousness. An additional dose is given every 6-12 hr for the next 48 hr upon signs of discomfort or pain. Wound clips are removed at 12-14 days post-surgery. The recipients are then housed together for ˜60 days prior to initiating breeding studies. In some embodiments, the male recipient are housed for between about 45 and 75 days. In some embodiments, the recipient males transplanted with spermatogonial lines are housed for no more than 45 days. In some embodiments, the recipient males transplanted with spermatogonial lines are housed for no more 50 days. In some embodiments, the recipient males transplanted with spermatogonial lines are housed for no more than 55 days. In some embodiments, the recipient males transplanted with spermatogonial lines are housed for no more than 60 days. In some embodiments, the recipient males transplanted with spermatogonial lines are housed for no more than 65 days. In some embodiments, the recipient males transplanted with spermatogonial lines are housed for no more than 70 days.

Recipient males transplanted with spermatogonial lines are paired with wild-type female Sprague Dawley rats of similar age at 60-70 days post-transplantation. Typically, the first F1 progeny are born between 100 and 150 days post-transplantation and recipients can continue to sire litters for greater than 300 days post-transplantation due to the long-term spermatogenesis colony forming potential of laminin-binding rat spermatogonia. Transgenic rat progeny from recipient-founders and wild-type females are identified by genomic PCR and/or Southern Blot analysis using probes specific to the mutation of interest.

### Stem Cell Technologies

### Spermatogonial Stem Cells (SSCs)

Site-specific genetic modification to sperm cell progenitors prior to differentiation and development can be carried out by modifying spermatogonial stem cells (SSCs) which develop into spermatozoa through the process known as spermatogenesis. Site-specific genetic modification of enriched SSCs is possible in vitro through use of various site specific genetic modification technologies. Transplantation of SSCs containing site-specific mutations into the seminiferous tubules of bulsulfan treated and/or genetically sterile male rats lacking the germ-line specific gene product DAZL results in maturation of SSCs into genetically modified spermatids. The genetically modified germ line recipient males are then bred with wild type females to produce offspring that harbor the site specific mutation (Production and Use of Rat Spermatogonial Stem Cell Lines (PCT/US2009/066275, WO/2010/065550, which are incorporated by reference in their entireties). In some embodiments, the invention relates to a composition or organism comprising one or more stem cells or one or more embryos, wherein the one or more stem cells or one or more embryos comprise one or more of the following mutations: (i) a deletion mutation; (ii) a knockout mutation; and/or (iii) an addition of a heterologous nucleic acid sequence; wherein the one or more mutations of (i), (ii), and/or (iii) are site-specific mutations caused by a Xanthomonas TAL nuclease (XTN) exhibiting at least about 1%, 2%, 3%, 4%, or about 5% mutation frequency upon its exposure to a genome of the one or more stem cells or embryos. In some embodiments, the invention relates to a method of mutating one or more stem cells or one or more embryos comprising the step of exposing a genome of cell with one or more XTN. In some embodiments, the invention relates to a method of mutating one or more stem cells or one or more embryos comprising the step of exposing a genome of cell with one or more XTN. In some embodiments, the invention relates to a method of mutating one or more stem cells or one or more embryos comprising the step of exposing a genome of cell with one or more XTN exhibiting at least about 1%, 2%, 3%, 4%, or about 5% mutation frequency upon its exposure to the genome.

### Embryonic Stem (ES) Cells

Embryonic stem cells are a pluripotent cell derived from the inner mass of the blastocyst or early stage embryo. Genetically modified ESCs from a donor are microinjected into a recipient blastocyst. Recipient blastocysts containing genetically modified ES cells are implanted into pseudopregnant surrogate females. The progeny, some of which have a genetic modification to the germline can then be established, and lines homozygous for the genetic modification can be produced by interbreeding.

Induced Pluripotent Stem (iPS) Cells

Induced pluripotent stem cells are artificially derived pluripotent cells from a less or non pluripotent cell, typically a somatic cell. There are multiple methods for which iPS cells can be “reprogrammed” to a pluripotent state from non pluripotent cells, including the expression of reprogramming factors. Genetically modified iPS cells from a donor are microinjected into a recipient blastocyst. Recipient blastocysts containing genetically modified ES cells are implanted into pseudopregnant surrogate females. The progeny, some of which have a genetic modification to the germline can then be established, and lines homozygous for the genetic modification can be produced by interbreeding.

### Somatic Stem Cells

Somatic stem cells or adult stem cells are potent cells found in organs after embryonic development. Somatic stem cells can be isolated from organs and tissues and have the potential to differentiate into many cell types of that organ and organism. For example, cord blood stem cells can be isolated from umbilical cord blood (CBEs) (McGuckin et al. (2008) Nature Protocols. 3, 6, 1046-1055). These cells are then expanded and used in the production of genetically modified organisms. CBEs are known as “embryonic-like” due to the expression of similar markers as embryonic stem cells. CBEs are a very small fraction of the cells present in umbilical cord blood. The CBE fraction is depleted of hematopoietic stem cells which stimulate hematopoietic commitment. CBEs are plated at high concentrations (10 million cells per 1 ml) in TPOFLK medium which is supplemented with extracellular matrix (ECM) proteins. The ECM proteins are essential for cell survival and aggregate formation similar to embryoid bodies which promotes cell-cell interactions and secretion of growth factors. Dynamic cell culture conditions are maintained based on cell phenotype: formation of floating aggregates, size and number of cell aggregates, cell adhesion and differentiation.

Genetically modified somatic stem cells from a donor are microinjected into a recipient blastocyst. For example, fresh or frozen cleavage stage embryos, produced from in vitro fertilization (IVF) can be cultured to blastocyst stage. The inner cell masses are isolated to produce ES cell lines that are capable of undifferentiated proliferation in vitro. Recipient blastocysts containing genetically modified somatic stem cells are implanted into pseudopregnant surrogate females. The progeny, some of which have a genetic modification to the germline can then be established, and lines homozygous for the genetic modification can be produced by interbreeding. Alternatively, genetically modified somatic stem cells can be reprogrammed into iPS cells in order to produce genetically modified organisms.

### Embryos

An embryo is a multicellular diploid eukaryote in early stage of development. Embryos can be genetically modified in vitro or in vivo. Embryos containing site-specific mutations may be implanted into pseudopregnant surrogate females. The progeny which have a genetic modification to the germline can then be established, and lines homozygous for the genetic modification can be produced by interbreeding.

### Methods

The methods used in the present invention are comprised of a combination of genetic introduction methods, site-specific genetic modification or mutagenesis mechanisms of stem cells, and generation of site-specific genetically modified organisms from the stem cells. For all genetic modification or mutagenesis mechanisms one or more introduction and delivery method may be employed. The invention may include but is not limited to the methods described below.

### Genetic Introduction Methods

In one introduction method, the site-specific mutation is produced in a stem cell. These stem cells can proliferate as cultured cells and be genetically modified without affecting their ability to differentiate into other cell types, including germ line cells. In the case of embryonic stem cells, genetically modified stem cells from a donor are microinjected into a recipient blastocyst, or in the case of spermatogonial stem cells, genetically modified cells can be injected into the rete testis of a recipient animal. Recipient genetically modified blastocysts are implanted into pseudopregnant surrogate females. The progeny, some of which have a genetic modification to the germ line can then be established, and lines homozygous for the genetic modification can be produced by interbreeding.

In one embodiment, spermatogonial stem cells (SSCs) with site specific mutations are used to generate genetically modified organisms. Preparing SSCs for site-specific genetic modification involves preparing feeder cell lines, and sub-culturing SSC lines. Preparing feeder cells may be carried out by thawing embryonic fibroblasts (EF), and placing on gelatin coated surface in SSC feeder medium. Sub-culturing SSCs may be carried out by seeding SSCs on EF medium. A 1:1 to 1:2 split passage is required before expanding into larger SSC numbers. Once established after the first several passages on EFs, cultures of spermatogonia are passaged at ˜1:3 dilutions onto a fresh monolayer of EFs. For passaging, cultures are first harvested by gently pipetting them free from the EFs. After harvesting, the “clusters” of spermatogonia are dissociated by gentle trituration. Spermatogonia are easily distinguished during counting as the predominant population of smaller, round cells with smooth surfaces, as compared to occasionally observed, larger and often irregular shaped irradiated EFs.

### Site-Specific Genetic Modification or Mutagenesis Methods

The invention pertains to a site-specific mutation generated in a stem cell, which includes but is not limited to somatic stem cells, spermatogonial stem cells (SSCs), embryonic stem (ES) cells, embryos, and induced pluripotent stem (iPS) cells. Stem cells containing site-specific mutations are used to produce a genetically modified organism.

Generating site-specific mutations in stem cells, which can then be used to produce a genetically modified organisms first involves the design and development of a protein such as a XTN whose DNA binding domain is engineered for a specific target site within the genome. A protein consisting of both a DNA binding domain and a cleavage or insertional mutagenesis domain is developed.

In one embodiment of the invention, a site-specific mutagenesis technology is expressed in SSCs generating site-specific mutations. The binding domains of the site-specific mutagenesis technologies are modified to bind a particular location in the genome. The site-specific mutagenesis technology may be introduced into SSCs via transfection using lipofetamine. A transfection mixture may be prepared by mixing transfectamine with the site specific mutagenesis technology XTNs. After harvesting undifferentiated SSCs, one may then add transfection mixture to the cell suspension, incubate, wash and plate the SSCs onto fresh EF feeder layers.

In some embodiments, the stem cells of the present invention comprise one or more transposons, one or more inverted tandem repeats (ITRs) (shown in table 3) of a transposon of variants thereof.

In some embodiments, the present invention comprise one or more transposons, one or more inverted tandem repeats (ITRs) of a transposon wherein the variant sequence inverted tandem repeats are at least 70, 75, 80, 85, 90, 95, 96, 97, 98, 99% homologous to known ITRs and known transposon elements including but not necessarily limited to those ITRs shown in table 3.

In one embodiment of the invention, clonal selection of SSCs containing site-specific mutations may be carried out by first plating treated spermatogonia. The genetically modified SSCs are allowed to proliferate in cell number by replenishing the medium with fresh EFs. Selection for genetically modified SSCs may be carried out in several methods. Selection using a reporter gene or selectable marker and, followed by culturing with an antibiotic or cell sorting. Specific mutations may be identified by selecting clones, isolating DNA and DNA sequencing.

Screening for XTN mediated site specific modification such as knockout mutations via NHEJ or knockin mutations using homologous recombination (HR) is done by selection with co-transfected vectors. SSCs are co-transfected with a XTN and a selection marker vector such as a fluorescent marker or antibody resistance within a lipid-based transfection reagent. 1 ug total DNA is transfected with a ratio of 500 ng XTN to 500 ng selection vector. Clones are isolated and propagated to sufficient numbers to isolate DNA for screening and sequencing.

Generation of Genetically Modified Organisms from Stem Cells Containing Site-Specific Mutations

The invention pertains to a site-specific mutation generated in a stem cell, which includes but is not limited to somatic stem cells, spermatogonial stem cells (SSCs), embryonic stem (ES) cells, induced pluripotent stem (iPS) cells and embryos. Stem cells containing site-specific mutations are used to produce a genetically modified organism.

In another embodiment, SSCs containing site-specific mutations are generated to produce genetically modified rats. The method for producing such organisms involves germline transmission of the genetically modified SSCs. Wild type and genetically sterile DAZL deficient organisms are prepared for transplantation of SSCs containing site-specific mutations into seminiferous tubules of the testes. A cellular suspension of SSCs containing site-specific mutations is transferred to a sterile microfuge. Genetically sterile recipients are placed in the supine position. The abdominal skin is then opened just rostral to the pelvis, and the testis exposed. The efferent ductules leading into the rete testis are then accessed by blunt dissection using micro-dissection forceps. The ductules are further dissected up to the base of their respective testis to yield visible access to the rete, which will be the site of injection. The injection needle holding SSCs containing site-specific mutations is manually inserted into the rete of the testis, and the cells are transferred into the testis by injection. The injected testis is then carefully placed back into the abdominal cavity and the same procedure can be performed on the contra-lateral testis to achieve more optimal breeding. Once injected and placed back in the abdominal cavity, the abdominal wall (sutured) and skin (wound clips) are surgically closed. Recipient males transplanted with SSCs containing site-specific mutations are mated with wild-type females to produce genetically modified organisms.

In some embodiments, mating between DAZL deficient males carrying genetically modified sperm and wild type females will produce progeny with approximately 50% of the offspring being heterozygous for the site specific genetic modification. Breeding and maintaining the colony involves PCR genotyping to identify which animals harbor the mutation. Once the animals are identified proper genetic crosses can be set up to produce numbers of homozygous, heterozygous and wilt type littermates.

In some embodiments, the invention relates to a composition comprise one or more stem cells or one or more embryos and one or more XTN. In some embodiments, the invention relates to a composition comprise one or more stem cells or one or more embryos; and one or more XTN; and, optionally culture media for the one or more stem cells or embryos.

### EXAMPLES

### Example 1

### Generation of Knockout Rats: Gene Disruption Technique Targeting the Rag 1 Gene (Prophetic)

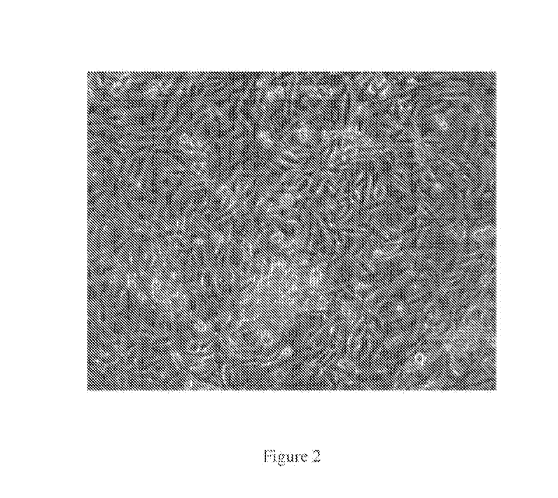

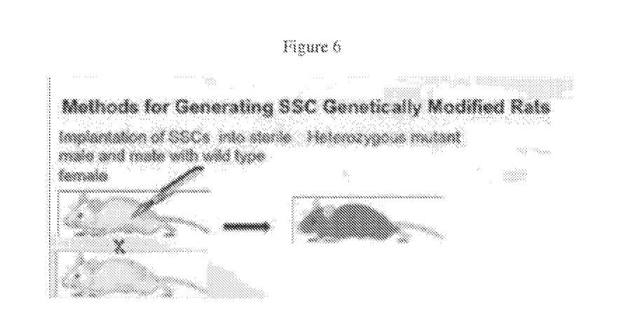

XTN technology can specifically bind and cleave designated DNA sequences for mutation of the targeted sequence. A schematic of wild type SSCs in colony are shown in FIG. 2. Site-specific XTN will be used to genetically modify rat spermatogonial stem cells (SSCs). In one example, the site-specific technology using XTN will be employed. XTN DNA binding domains can be engineered to bind to a sequence of choice. The XTN binding domain will be engineered to bind to the rat Rag1 gene, proposed binding and mutation sites as well as XTN sequences are shown in FIG. 5. The rat Rag1-specific XTN will be expressed in rat spermatogonial stem cells (SSCs) along with a selection marker (e.g. fluorescent marker or homologous recombination vector) which will indicate that the XTN construct was successfully transfected into the cell. FIG. 4 displays a schematic of wild type SSCs as well as SSCs that will be transfected and selected for the XTN construct and the selectable marker. The XTN and selectable marker co-transfection will result in site-specific double-stranded DNA breaks followed by NHEJ repair. Rat SSC clones will be sorted and mutation screening will be used to identify knockout clones. Propagation of XTN-mediated genetically modified rat SSCs in order to generate ample numbers for recipient injection is shown in schematic FIG. 3. Genetically modified SSCs will be expanded and germline transmission will be carried out in the method described above to produce genetically modified Rag1 knockout rats (FIG. 6).

### EXAMPLE 2

### Generation of Nrf2-Mutations in Spermatogonial Stem Cells

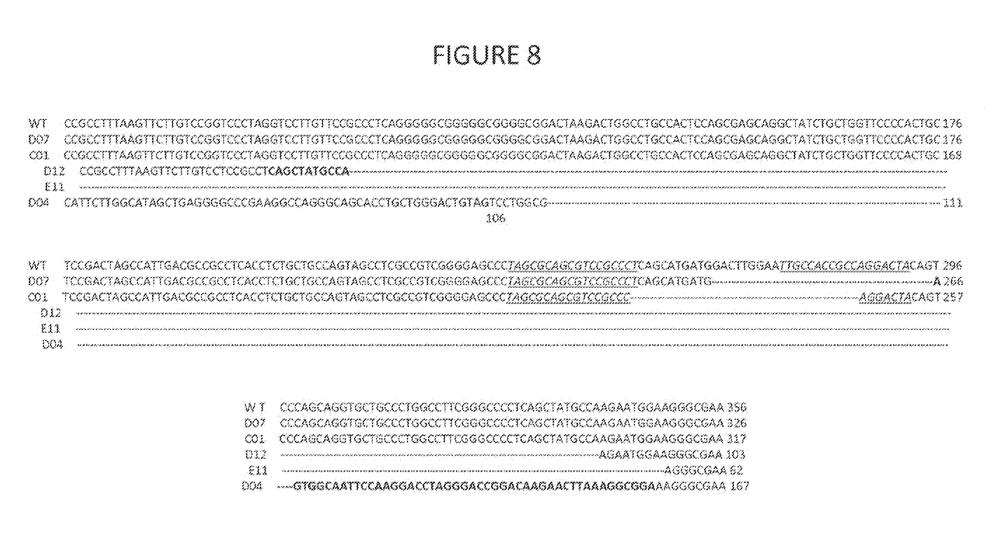

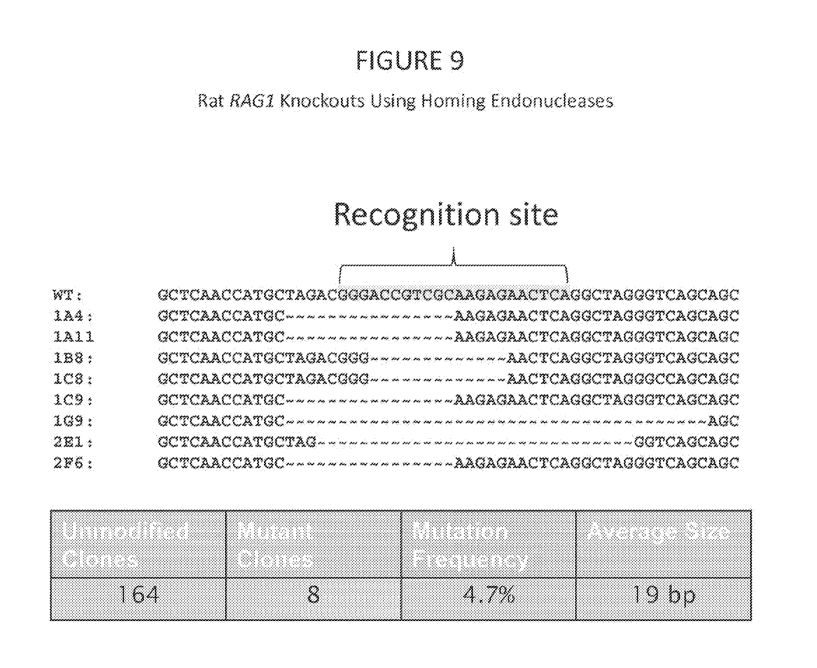

2×10⁶ rat SSCs were transfected with approximately 30 milligrams of a Nrf2-specific XTN pair. The cells were co-transfected with a DsRed-neo plasmid, which expresses a red fluorescent marker gene and a neomycin resistance gene. The cells were allowed to incubate in culture for 120 hours before isolation of clones. DsRed-neo positive cells were isolated by selecting for G418^{R} cells (selection for neo-positive cells). DNA from the stem cells were amplified using primers flanking the XTN-binding site and individual products were cloned for sequence analysis for the presence of non-homologous junction points (NHEJ). PCR products generated from Nrf2 XTN-transfected cells were shotgun TOPO cloned and 90 clones were sequenced. 86 good quality reads were generated and compared against the wild type sequence for Nrf2. Five clones of the 86 generated clones demonstrated an NHEJ event which is equivalent to about 6% frequency (FIG. 8). Clone C01 has a 30 bp deletion and clone D07 contains a 72 bp deletion. Clone D12 appears to have a 246 bp deletion. XTN target sites are in bolded in FIG. 8, while disrupted or mutated sequences are italicized. Three sets of parallel experiments using homing endonucleases were run as experiments for comparison against the XTN pair detailed above. In a first experiment, 1×10⁶ rat ES cells were transfected with 2 milligrams of a plasmid expressing a homing endonuclease engineered to recognize a sequence within rat exon 2 of Rag1. Similar to above, the cells were co-transfected with a DsRed-neo plasmid which expressed a red fluorescent marker. Cells were allowed to incubate for 120 hours. DsRed positive cells were isolated by cell sorting five days after transfection. 96 positive clones were selected by FACS sorting technique well known in the art. DNA was extracted from the 96 positive clones and PCR amplified using primers specific for the exon 2 DNA segment containing the Rag1 gene. Individual DNA sequences were analyzed for sequence analysis. None of the 96 clones selected contained a NHEJ.

In a second experiment, 2×10⁶ rat SSCs were transfected with either: (a) 3 mg or 30 mg of RNA in vitro synthesized from the Rag1-specific homing endonuclease (no polyA tail); or (b) 3 mg or 30 mg of RNA in vitro synthesized from the Rag1 specific homing endonuclease (with a polyA tail). Each group was co-transfected with the same DsRed-neo plasmid as detailed above. DsRed positive cells were isolated by cell sorting five days after transfection. 384 different cellular clones were selected and DNA was isolated using well known DNA isolation techniques. Exon 2 sequences from the DsRed positive clones were amplified by PCR using the same exon 2-specific primers and individual products above. None of the 384 clones displayed any NHEJ sequences.

For comparative purposes, a third experiment was performed in which 2×10⁶ rat SSCs were transfected with: 3 mg or 30 mg of RNA in vitro synthesized from the Rag1 specific homing endonuclease (with a polyA tail) with relevant control experiments. Each experimental group was co-transfected with the DsRed-neo plasmid as detailed above. DsRed-neo positive cells were isolated by selecting for G418^{R} cells (selection for neo-positive cells) and Exon 2 sequences were amplified by PCR using the same primers described above and individual products were cloned for sequence analysis using the techniques described above. Zero clones out of 182 analyzed incorporated any mutation.

In contrast to the homing endonucleases which displayed a zero frequency of mutagenesis in SSCs, the XTN pair successfully created clones of SSCs with a frequency of about 5%. Mutation frequency via NHEJ occurred at frequencies of ˜5% in rat ES cells, using homing endonucleases, however, no mutations via NHEJ were observed in 662 clones sequence analyses.

### EXAMPLE 3

### Generation of Knockout Minipigs: Gene Disruption Technique Targeting the Rag1 Gene (Prophetic)

Examples include using the site-specific Xanthomonas TAL Nuclease (XTN) technology, which can specifically bind and cleave designated DNA sequences for mutation of the targeted sequence. Site-specific XTN can be used to genetically modify minipig spermatogonial stem cells (SSCs).

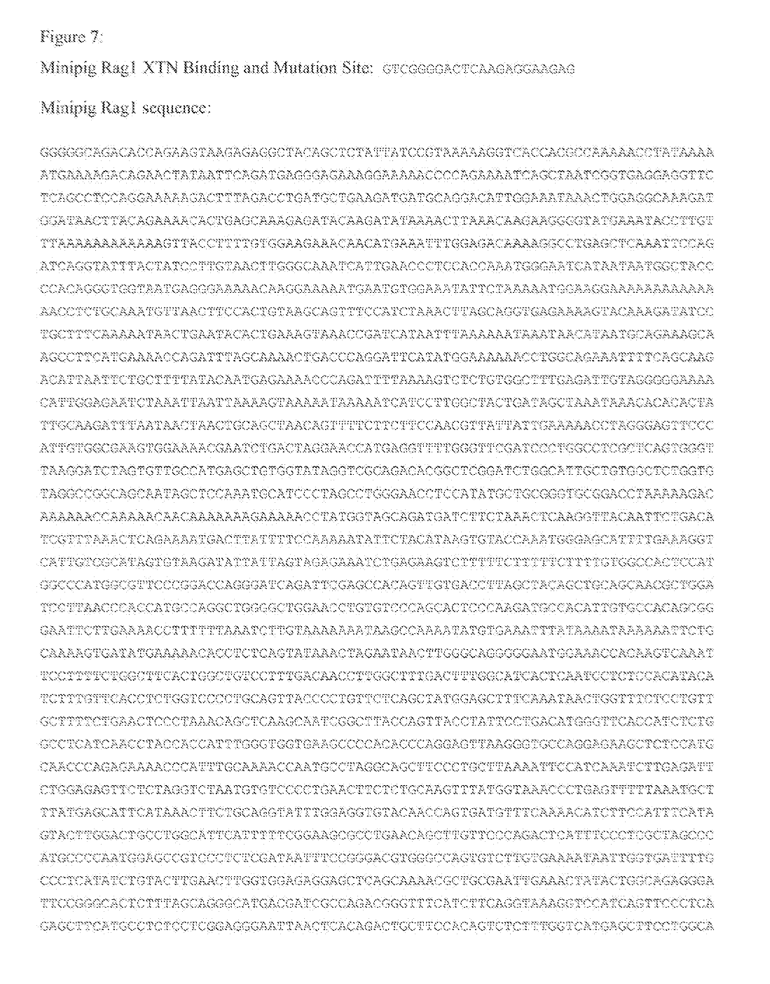

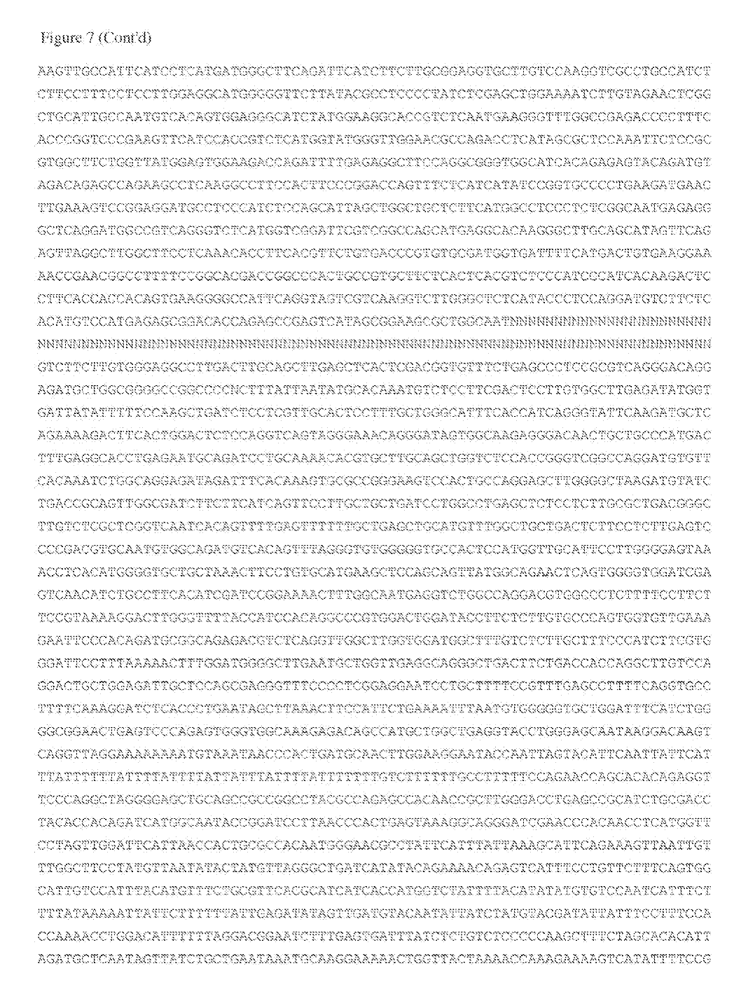

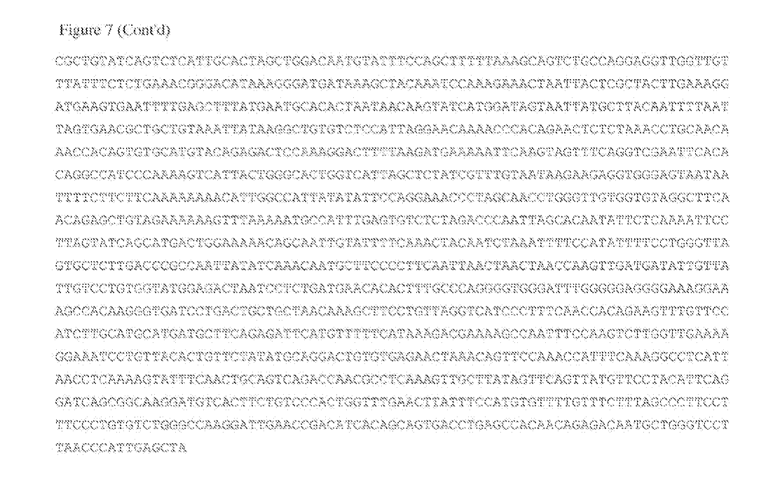

In one example, the site-specific technology using Xanthomonas TAL Nuclease (XTN) will be employed. XTN DNA binding domains can be engineered to bind to a sequence of choice. The XTN binding domain will be engineered to bind to the minipig Rag1 gene. The minipig Rag1 sequence as well as a proposed XTN binding site is shown in FIG. 7. The minipig Rag1-specific XTN will be expressed in minpig spermatogonial stem cells (SSCs) along with a selection marker (e.g. fluorescent marker or homologous recombination vector) which indicates that the XTN construct was successfully transfected into the cell. The XTN and selectable marker co-transfection will result in site-specific double-stranded DNA breaks followed by NHEJ repair. Minipig SSC clones will be sorted and mutation screening will identify knockout clones. Propagation of XTN-mediated genetically modified minipig SSCs is required in order to generate ample numbers for recipient injection and germline transmission. Genetically modified SSCs will be expanded and germline transmission will be carried out in the method described above to produce genetically modified Rag1 knockout minipigs.

Table 1 includes reagents for the production of SSC medium.

Table 2 includes reagents for passaging and propagation of SSCs.

Table 3 depicts Transposon inverted terminal repeats (ITRs)

TABLE 1
Reagents for SSC culture medium (SG Medium)
SG Medium

| | 500 mL | | 250 mL | | 200 mL | | 100 mL | | 50 mL | |
|---|---|---|---|---|---|---|---|---|---|---|
| DHF12 - Sigma D8437 | 469.5 | mL | 234.75 | mL | 189.8 | mL | 93.9 | mL | 46.95 | mL |
| 100 ug/mL rR-GDNF | 100 | uL | 50 | uL | 40 | uL | 20 | uL | 10 | uL |
| 25 ug/mL rbH-FGF | 400 | uL | 200 | uL | 160 | uL | 80 | uL | 40 | uL |
| Diluted 2-mercaptoethanol -Sigma | 5 | mL | 2.5 | mL | 2 | mL | 1 | mL | 500 | uL |
| M3148 | | | | | | | | | | |
| l-glutamine (100x) - Invitrogen 25030- | 10 | mL | 5 | mL | 4 | mL | 2 | mL | 1 | mL |
| 149 | | | | | | | | | | |
| B27 Supplement Minus Vitamin A (50x) - | 10 | mL | 5 | mL | 4 | mL | 2 | mL | 1 | mL |
| Invitrogen 12587-010 | | | | | | | | | | |
| Antibotic/mytotic (100x) - Invitrogen | 5 | mL | 2.5 | mL | 2 | mL | 1 | mL | 500 | uL |
| 15240062 | | | | | | | | | | |

TABLE 2
Passaging SSCs

| | Growth Area | Splitting | Nominal Fill |
|---|---|---|---|
| Size of Culture Vessel | (cm²) | SSC density | Volume |
| 96 well | 0.32 | 9.6 × 10\hat{ }3 | 200 ul |
| 8 well chambered coverslip | 0.8 | 2.4 × 10\hat{ }4 | 400 ul |
| 24 well | 1.88 | 5.6 × 10\hat{ }4 | 500 ul |
| 12 well | 3.83 | 1.2 × 10\hat{ }5 | 1.0 ml |
| 6 well | 9.4 | 2.8 × 10\hat{ }5 | 2 ml |
| 35 mm | 8 | 2.4 × 10\hat{ }5 | 2 ml |
| 60 mm | 21 | 6.3 × 10\hat{ }5 | 5 ml |
| 10 cm | 55 | 1.7 × 10\hat{ }6 | 10 ml |
| Flasks: | 25 | 7.5 × 10\hat{ }5 | 5 ml |
| | 75 | 2.3 × 10\hat{ }6 | 10 ml |

TABLE 3
Transposon ITRs

Transposon
Sleeping Beauty
5′ Inverted Tandem Repeat:
CAGTTGAAGTCGGAAGTTTACATACACTTAAGTTGGAGTCATTAAAACTC
GTTTTTCAACTACTCCACAAATTTCTTGTTAACAAACAATAGTTTTGGCA
AGTCAGTTAGGACATCTACTTTGTGCATGACACAAGTCATTTTTCCAACA
ATTGTTTACAGACAGATTATTTCACTTATAATTCACTGTATCACAATTCC
AGTGGGTCAGAAGTTTACATACACTAAGT
3′ Inverted Tandem Repeat:
ATTGAGTGTATGTAAACTTCTGACCCACTGGGAATGTGATGAAAGAAATA
AAAGCTGAAATGAATCATTCTCTCTACTATTATTCTGATATTTCACATTC
TTAAAATAAAGTGGTGATCCTAACTGACCTAAGACAGGGAATTTTTACTA
GGATTAAATGTCAGGAATTGTGAAAAAGTGAGTTTAAATGTATTTGGCTA
AGGTGTATGTAAACTTCCGACTTCAACTG
piggyBac
5′ Inverted Tandem Repeat:
CCCTAGAAAGATAGTCTGCGTAAAATTGACGCATGCATTCTTGAAATATT
GCTCTCTCTTTCTAAATAGCGCGAATCCGTCGCTGTGCATTTAGGACATC
TCAGTCGCCGCTTGGAGCTCCCGTGAGGCGTGCTTGTCAATGCGGTAAGT
GTCACTGATTTTGAACTATAACGACCGCGTGAGTCAAAATGACGCATGAT
TATCTTTTACGTGACTTTTAAGATTTAACTCATACGATAATTATATTGTT
ATTTCATGTTCTACTTACGTGATAACTTATTATATATATATTTTCTTGTT
ATAGATATC (minimal sequence is underlined and
bold, i.e., first 35 bp)
3′ Inverted Tandem Repeat:
TAAAAGTTTTGTTACTTTATAGAAGAAATTTTGAGTTTTTGTTTTTTTTT
AATAAATAAATAAACATAAATAAATTGTTTGTTGAATTTATTATTAGTAT
GTAAGTGTAAATATAATAAAACTTAATATCTATTCAAATTAATAAATAAA
CCTCGATATACAGACCGATAAAACACATGCGTCAATTTTACGCATGATTA
TCTTTAACGTACGTCACAATATGATTATCTTTCTAGGG
(minimal sequence is underlined and bold,
i.e., first 35 bp)

1. A composition comprising one or more stem cells or one or more embryos, wherein the one or more stem cells or one or more embryos comprise one or more of the following mutations: (i) a deletion mutation; (ii) a knockout mutation; and/or (iii) an addition of a heterologous nucleic acid sequence; wherein the one or more mutations of (i), (ii), and/or (iii) are site-specific mutations caused by a Xanthomonas TAL nuclease (XTN).

2. The composition of claim 1, wherein the heterologous nucleic acid sequence is chosen from a selectable marker or an orthologous gene.

3. The composition of claim 1, wherein the one or more stem cells is chosen from a spermatogonial stem cell (SSC), an embryonic stem cell, or an induced pluripotent stem cell.

4. The composition of claim 1, wherein the one or more stem cells is derived from the germline lineage of an animal or plant.

5. The composition of claim 1, wherein the one or more stem cells or the one or more embryos further comprise at least one inverted tandem repeat of a transposon or a variant thereof.

6. The composition of claim 1, wherein the one or more stem cells is a somatic stem cell.

7. An organism comprising one or more stem cells, wherein the one or more stem cells comprise one or more of the following mutations: (i) a deletion mutation; (ii) a knockout mutation; and/or (iii) an addition of a heterologous nucleic acid sequence; wherein the one or more mutations of (i), (ii), and/or (iii) are site-specific mutations caused by a XTN.

8. The organism of claim 7, wherein the one or more stem cells comprises an SSC.

9. The organism of claim 7, wherein the one or more stem cells further comprise at least one inverted tandem repeat of a transposon or variant thereof.

10.-15. (canceled)

16. The organism of claim 7, wherein the composition is a colony of mammals.

17.-19. (canceled)

20. The organism of claim 7, wherein the organism is chosen from a mouse, pig, rabbit, dog, cat, goat, non-human primate, mini pig, ferret, farm animals, fish, chicken, and bird.

21. The organism of claim 7, wherein the organism is a plant chosen from: rice, tobacco, wheat, potato, soybean, tomato, Arabidopsis, maize.

22.-30. (canceled)

31. A colony of genetically modified organisms comprising:
(a) at least one organism comprising one or more stem cells, wherein the one or more stem cells comprise one or more of the following mutations: (i) a deletion mutation; (ii) a knockout mutation; and/or (iii) an addition of a heterologous nucleic acid sequence; wherein the one or more mutations of (i), (ii), and/or (iii) are site-specific mutations caused by a XTN.; and
(b) progeny of the organism of subpart (a).

32. The colony of claim 30, wherein the heterologous nucleic acid is a selectable marker or an orthologous gene.

33. The colony of claim 30, wherein the at least one organism and the progeny further comprise at least one inverted tandem repeat of a transposon or variant thereof.

34. The colony of claim 30, wherein the at least one organism and the progeny further comprise a nucleic acid that comprises a transposon sequence that is at least 70% homologous to: SEQ ID NO:1, SEQ ID NO:2, SEQ ID NO:3, and/or SEQ ID NO:4.

35. A method of generating one or more genetically modified organisms comprising:
(a) contacting at least one stem cell derived from the germline lineage of an animal or plant by the stem cell with: (i) at least one XTN that mutates a gene of interest; or (ii) at least one expression vector that encodes a XTN that mutates a gene of interest, thereby creating at least one stem cell comprising at least one mutation at a gene of interest;
(b) expanding an in vitro culture of the at least one stem cell comprising at least one mutation at a gene of interest;
(c) implanting one or more stem cells from the culture of step (b) into an organism.

36. The method of claim 35, wherein the organism is capable of passing at least one mutation at a gene of interest to progeny by germline transmission.

37. The method of claim 35, wherein the genetically modified organism is a mammal.

38. The method of claim 35, wherein the genetically modified organism is a rat or mini pig.

39. The method of claim 35, wherein the genetically modified organism is a sterile male rat or sterile male mini pig.

40. The method of claim 35, wherein the method further comprises: breeding the organism implanted with the one or more stem cells with another animal to generate one or more progeny that comprise the mutated gene of interest.

41. The method of claim 40, wherein the progeny are mammals.

42.-44. (canceled)

45. A method of manufacturing a first filial generation of genetically modified organisms comprising two or more distinct subsets of organisms, the method comprising:
(a) contacting a first stem cell with: (i) a XTN that mutates a first gene of interest; or (ii) an expression vector that encodes a XTN that mutates a first gene of interest; thereby creating a first stem cell comprising a first mutation;
(b) contacting a second stem cell with a modifying agent, thereby creating a second stem cell comprising a second mutation;
(c) expanding an in vitro culture of each of the first and the second stem cells;
(d) implanting a mixed population of stem cells comprising the first and the second stem cells into an organism;
(e) breeding the organism with another organism of the same species.

46. The method of claim 45, wherein the first filial generation of genetically modified organisms comprises two or more sets of mutated genes of interest, each set of mutated genes of interest comprising a distinct mutation of interest derived from a haplotype of distinct stem cells transplanted into a parent of the organism.

47. The method of claim 45, wherein at least one stem cell of the mixed population is a spermatogonial stem cell of a mammal.

48. The method of claim 45, wherein the organism is a mammal.

49. (canceled)

50. A kit comprising:
(a) the composition of claim 1; and, optionally
(b) culture media for the one or more stem cells or one or more embryos.


SEQUENCE LISTING (35 sequences)

SEQ ID NO: 1 - DNA, 229 bp, Artificial Sequence
  Partial Sleeping Beauty Sequence 1
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagt

SEQ ID NO: 2 - DNA, 229 bp, Artificial Sequence
  Partial Sleeping Beauty Sequence 2
attgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 3 - DNA, 309 bp, Artificial Sequence
  Partial piggyBac Sequence 1
ccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatc

SEQ ID NO: 4 - DNA, 238 bp, Artificial Sequence
  Partial piggyBac Sequence 2
taaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctaggg

SEQ ID NO: 5 - protein, 6 aa, Artificial Sequence
  Homology Peptide 1
ACDEFG

SEQ ID NO: 6 - protein, 6 aa, Artificial Sequence
  Homology Peptide 2
ACDHIK

SEQ ID NO: 7 - DNA, 11097 bp, Rattus norvegicus
  misc_feature (8598)..(8599): n = a, g, c, or t
ttgctatctctgtggcatcgaatattaacaaccaaactgcagacattctaacactctggccgggaggcctgtggagcaaggtaactcttgtgcttttcatgccttgagccaaaatattcattatgtttttgtgtttctaatcagaactatgagagatggtttccttgcaggcattgaagggaggggagaggaaagctggaactgattttgcttttgtttggaatgaagaaaaactagaaagggggtaaaaggccaagttcagagacatggcaggcttatagaacaggtggaacacatctaagttgatataaatggatgctattggcagaggagggctcaagaagagcggcagaagcagagatgagctaacacactgttagctcaggcaagaaaaaaataataaccaggaacaaagaactccaggcagggaagaaatgagcagttttccagttttgacatttccccaactgagatgtctggtgtttcagaaaatgtgacctgtataccacttcatcaggaccactgcaatatttaaaaatgcaatttctggactccatcctaagcaaataggcagagtctctgggagctaatgatctgcctgctttggaaaacaatctccgtagtggttctgactcagattaattttgagaattgccatgggagagagcctactccatctaaggttctgctctcgggtgtgtaataaagggttgtaactctgcattgctttctaaagtggttcggtgaggggtcccctggaaggaggacgacaacttcctggttattttgtagatgcttgaaatgtttttgtgcatgatggggggcagacaatcaggagtggtacccgtggagagagcaaaggagtaggacaggtgtgacaaagagggatcacacttgcgtgcacagagtgtgactggagtggggagagtgcttagtcaacagagtgcttgcaagtgtggagatctgagtgggactccccaaagctttgtaaaacatctgggtgtgatattccattcttgtaatctccacaccagggagaaagaggcatggtctctggggcttactggccagtcagtgctgatctaactgttagcaggagagcgtgagtcacgcagtggagtgcacgtgttgcagttagggctgagatagaaatgaactgctgctaggctttctcgacatctcaggttactaatggtgtctcactacaagtcacagcattgcctcatccgtaccaccccaggctggtttcacacagagtccatggcctatggaaggagggacattacagtcgtggtgacagatgcttacaacactgaggtgatgctgctgactgggataagctgagccagcatcagaccctcagcctttagactctgaggtgtctactctcagacctattgctgcaagagaggacactgagcatttgaaggatttcaaatcacccttccatggtcctcgcttcttgttgtcttcaggttggattttagtcccccctgagacatatttattagaagacaaacaaacaaacaaaaaaagaataaagatgctggtcaggtttaagacttggtaggtcttaaactcctttggagatcaattaaaaagaaagaatgtaaaaaagaccttgagaacctacatatctggtcttaaaaatgtttcatttgagctgaattgatcatatggaaacttcaaataaaaaaataataaaattaacagacatttttcttagttctaattgtactttctggtgattaagcactattaaggttttagtgcaccaatttctagaattctcatctttctgtgtgtacaaatataaatatgtatgcataagcaggatcctgctgtacttgctttgtttctgtcttaccttcctgcacatggacttcttttgtttacttctgtctatagaaatcacacatatgtgtaatatatgcataatgtagtttctagtctctgtctgcttttcaaagtggctaataacctttcaaataaatatccccaggcattatctaaaaattatcatatacttgtggaggtatttctatagaataactatagaatatttccccaaaggaaatttatcacaccaaagacaacacatcttaacatttaaaaggctttactaaagtgtcactgtgctcttttactctcacgctactctcaagggtatatgaaagtgtcttgatttcttcatgccttcattaaaactaaatgataccaatagttttaatctttgataattggagttgttacaaggactcactaaatcagattttttaaagttctatcttttcccaaactcattaaacatgtagcctccacagaaactcccattaacatccttgtacatagtcccttaaaacatatcatgtgcctgctatctccattttgtttcaatgtttcagtacatttttatgatttgagttgtaaagcaacctaaaaactacatttgatttgactggccatagctagtaactatctcattccatggggacatttcaacagatgtgattggtgggtttggtcatgatgtttcaggttcgaggaagtcaggggaggttgacttgctgaatgtgaaaggccaatatttaatgtgttgaaaatggcatttggttcaagccaaaggcaaggaacacagagattggctttgcatttgttacaagacagaatgaagaaacaattgatcaggaaacaactttgaggcctttgcttggttgcacttgaaatacctttggggttcttgagacctttgaaatgtttctaggattgtttagctcccatagttctgcattaggaacagccaaactgtgtttctttatcaatcaagtcttagaaatttgctcttaattttaatgaagaaaaaaaccccctctgatgtgtaatacagatacacatagaataaagtatgttagtctaaagggaagaactcaaaaagcaatgttgctagcaggatccaggactagtacttacctccatcactctgttcctgatggccaaagtcccactcctggtcaaaatgtgttcatttgagacattaacttcctgactacatggctataaagctcactgagttggttaatctgttgaagggaaaatggatttctagacacaagttgtaggttaaaagagttaatccagacacagttgggaaatatcgatgagatggtatggtatgaactaggaaatgtaggaagtttggtctaatttgagagcattcagatgacaccatctactcttcttctacattgagtttgaaatttatattacaattggcatatttattgttgtttttactactattctttttaggtttatctattttctgtatatgagtacactataactaattactatcttcagacacaccagaagagggcatcagatcccattacagatggttgtgagacaactcgtggttgctaagagttgaactcaggacctctggaagagcagtcagtgttcttaaccactgagccatctctccagtcccctccttttactctttactcttattaatacttgagacagggtctcattatgtagcccagggtggcctagaactctctagggtcgaccaggctgttctcaaacttgtagtgattcttctgttcccacctctttagaactcgatttgtagacaaacaccatcattctcttctgtaattaaaaaaatatttctttactttttgaggttataatgcaaaccccatcatttccctcacctgtaatatttttaaccagaagaaagttgttcctcatatctcttctcattactaactgtagtgctaatgacctttggtctacagtgactttcaggatgacctgtgtgagttttgtctgaaaccacttgaagttttcatcttagaagtccttcatcttcttagcatgcttgtggtatggtttgctgctggtttaaagagttctgtggttctgtttctaatgtagactcagccttctaatttacagcggcattcgctaattaaaatcgtaagcgtaattattatccgtgatacttattattagtttgcattcataaagctcgaaattcactccatcctttcaagcagtgagtagttagtttcttgggtttgtagctttatcatctctttatgacccatttggaagaaataaacaaccaatctcctggaagaatgctttaaaaatctaaaaatatattgtccagttggtgtaatgaggctaatacaatttggtttagtatccagtttatcactgcatttacgcacaaacaactactgagcacctaataggtaccagggatattggtagatgaaatcaatcattcaaagattctgtcttcaaagatgttcaggtttttgtggaaagggaattagatttcacatatctatatatttaaaatcatgcatatatgtagtataagtgtccccaaatattatcaggaaaaggattgatcatttatggctgtacgatcggcacctaacagagcttagtaaaatagatttaatagctatctttggaaagaattaaatgaatgaaaatttgtgtgcattgatctcagttatttccagaatactgcaatatgatatgtatacttttcctttttgttttttgtttgtttgacacttttcttaacttgacttttccctccctccattttccccaggtagcttcgccaaaatggctgtccccttgccatctaccctgagactcagttctgcacctgatgaaattcagcacccgcacatcaaattttccgagtggaaatttaagctgtttagggtgagatcctttgaaaaggcacccgaagaagcacaaaaagagaaggattcctcagaagggaaaccttgtctcgagcagtctccagtagttctagataagcctgggggtcagaattcagttctgactcaacgagcattgaaactccatcctaaattttcaaagaaattccatgttgatgggaagtcaagcgacaaagcaattcaccaagccaggcttagacacttctgccgcatctgtggcaatcacttcaagagtgacgggcacaaccggagatacccagtccacgggcccgtggacgctaaaactcaaagccttttccgaaagaaggaaaaaagagtcacgtcctggccagatctcattgccagagttttccggattgatgtgaagtcagatgttgactccatccaccccactgaattctgccataactgttggagcattatgcacaggaagttcggcagtgctcacagtcaggtctactgcccaaggaatgtgaccgtggagtggcacccccacacaccgtcctgtgacatctgctttactgcccatcggggactgaagaggaagagacatcagcccaacgtgcagctcagcaagaaactaaaaactgtgctcaaccatgctagacgggaccgtcgcaagagaactcaggctagggtcagcagcaaggaggtcatgaagaagatctccaactgcagtaagattcatctcagtaccaagcttcttgcagtagacttcccagcacactttgtgaaatccatctcctgccagatatgtgaacacattctggccgatccggtggagaccacctgtaagcacctgttctgtagaatatgcattctccggtgcctcaaggtcatgggcagctattgtccctcgtgccgatatccatgcttccctactgacctgcagagtccggtcaagtcctttctgagcatcttgaattctctgatggtgaagtgtcctgcccaagagtgcaatgaggaagtaagtctggaaaaatataaccaccatgtctcaagccacaaagaatctaaagagactttggtgcatattaataaagggggccggcctcgccagcatctcctgtcactgacgcgaagggctcagaaacatcggctgagggagctcaagattcaagtcaaagaatttgcagacaaagaagaaggtggagatgtgaagtctgtgtgcttgacgttgttcctgctggcactgagggcaaggaatgagcacagacaagctgatgagctcgaggccatcatgcaaggccggggctccggtcttcaaccagctgtttgcttggccatccgtgtcaataccttcctcagctgtagccaataccataagatgtacaggacggtgaaagctatcactgggaggcagatttttcaacctttgcacgctcttcgcaatgctgagaaggtcctcctaccaggctaccacccctttgagtggcagccccctctgaagaacgtgtcctccagaactgacgttggaattattgatgggctctctggacttgcctcctccgtagatgaatacccagtggacacaattgcaaagaggttccgctatgactctgctttggtgtctgctttgatggacatggaggaagacatcttggacggcatgagatcccaagatcttgatgactacgtgagtggtcccttcaccgtggtggtaaaggaatcgtgtgatggaatgggggatgtgagtgagaagcacgggagtgggcccgcagttccagaaaaggcagttcggttctctttcaccgtcatgagagttacagtagagcatggctctcagaacgtgaagatatttgaggaacccaagcccaattctgaactgtgttgcaagccactgtgccttatgctggcagatgagtctgaccacgagacacttactgctattctcagccccctcatcgctgagagggaggccatgaagagcagtgaattgctgctggagatgggaggcatccccaggacttttaaatttatcttcaggggcactggatatgacgagaaacttgtcagggaagtagaaggcttggaagcttctggctcagtctacatttgtacactttgtgatgccacccgtctggaagcctcccaaaatcttgtcttccactccataaccagaagccacgccgagaatctgcagcgctatgaggtctggcggtccaatccctatcacgagtcagtggaagaactccgggaccgggtgaaaggagtctctgccaaacctttcatcgagacagtcccttccatagatgcgcttcactgtgacattggaaatgcagctgaattctataagattttccagctggagataggggaagtgtataaaaatcccaatgcctctaaagaggaaaggaagagatggcaggccaccttggacaaacatctgcggaaaaggatgaacttaaaaccaatcatgaggatgaatggcaactttgcccggaagcttatgacccaagagactgtggacgcagtttgtgagttaattccttctgaggagaggcacgaagctctcagggagctgatggacctttacctgaagatgaaaccggtgtggcgctcatcgtgtcctgctaaagagtgcccggagtccctttgtcagtacagtttcaactcacagcgtttcgctgagctcctctccaccaagttcaaatacagatacgagggcaaaatcaccaattactttcacaaaaccttggcacacgtccctgaaattattgaaagggatggctctattggggcatgggcaagtgagggcaatgagtctggtaacaaactgtttaggcgcttccggaaaatgaatgccaggcagtccaagtgctatgagatggaagatgtcctgaaacaccactggctgtatacttcaaaatacctccagaagtttatgaatgcccataatgcattaaaaaactctgggtttaccatgaactcaaaggagactttaggggactctttggacattgaggactctctggaaagccaagattcaatggagttttaaagttggtatttgcagatgtgttttcctttgggttgcagcgaggtcttctcttagcacctagcacattgttgtgtgggtgggtcttatcatccaggggtgacaggctggagtgagaggccagaggtgctgtcacctggtcagggattgatagttgatgagatgagattgcctgagtcgtttagtgaggccagaagagcaacaggagaaatcagttacttggaagctcaataacttgggacagcggtagcttcaggggaccagggatgcacagagatatgtgtgtgcatgccactgcgtgccatgaaaattaaagccagggctgtcaaaggacagccaagcatgctaggaaagaaatttgtcttgggtatgtcttttttctttttttccacctgaatttattatattttatattgagatataatgctggttttttggccatgtcatattttgtagaactattatgtaaatttcacgtatgttaatttattctgcaagattcgaaggagcctttgagttttctctaacaacactctccatttgtaggacatggtagccattaccaagggctcttagtgcattgtcttatgtgctacgtttatgcagaaataaattaaatcaagacaggtttcagaaaaacagccttttccaggtttaagtggcctcagtattgtcactttcagtgtctatgaggacatctggtgtcataaaattttcaatgatgaaggtagagaaccttttagctacagctacctattaagtatgataaactcgtgtccacttagctgtctagagccaactctggttgtttattttactttnntaattaagagtaatttatagtcttgtgtttttccatgtgatctcaaggttacagatttttaaaatccctatttccttactgcctgaaaacagatttaatacctgctgaaaatgtcagatttttctctatatgaaacatgggtcagtctgagtaagtccagggttcttaaaggcctctgtttctgctatgcgttattattttgattatgatcagttgagtttcagtgtgtgcagatcttagtatcggtacagtgctgcaccttttctatttgttacatttagatggagtctacttataatagagtagaggaatgaacatgtgtacattgtttatttccattttatcacttctggattttttgaattgtccctcaaattacagggatgatcaatacctgattttgaatttaaacccaagcctgtctcttaatctgggttctttatactgtgtttatgttgttcatatggtagcattagaatttatttaggataactaaccactgcagcttacaatcccgagagttatattttttgggatggaaagtcatctgtttgtcttgggcctttggatagaataaaattgggccaaacctctaaggaatccataaaccctccagtagctgcggttatgtggaaactatttatgtccttaggaattcagcgtttgccaggcaccagcaacttaagagcaggcatgggattttatttgctagtgagtcataatgatatgttagcgttaattagtttttttcttcctttgattttattggtcataattgtcagtcttcatacacagtatatcaaagagctttgtaatttagctgtcaagtgtgcaagagtgacattaccctaaatgacacacatttggggcaaatgcagggattgcatttaatgctttctactggaagatgcagccattttctttgctttctgtttggagcacatcgaagacattcataatttagttgtcaaaaacatgcacacgcttaagtattatcttaaaaaccctttttgttttgtcatttgcaaaccaattggttcctaaagcaccggtcctttcatcccctttaatctttacagatgtctgtgcactaataaacagctaagttcccaccgtcctcctgctccccagagcatgctggcactgttcggtagccgttttccccactcactctgttagtcaattggcagcactgtaaatactgcccaatattaaattccccttgctaggattgtaggacaatttcagaagcatttgactcagggcatgatttatagcacaatcttctcccgactctcctaatcagatcatattctgataaacctcaagaacacctggccacaggatttcaatatgtagagtgtcccctgtggctgtttgtttccctcagtgtggagactgcagtatcctcaaccctgctctagggatactctacttttatttttgagaaggcccattgcaaatgcaggcatattcttcagggaaattcaccccaacgaatgggggatttccccactgctccccccaacctccctccccgccagggctggagaagtgcaatacttttttttctgttctgaaacctgaaacctgtttctataggtcttgccagccttctgcactgcactgcagggaccagtgactgtggctgatgaaaattgatgaatggtgggtgagaggttaagaagtgtcttgggattaataaacatgcacacagatgccagagcagcggggaaagatgtctctcaggtggataggaatttaattttgtctcaatgtgttttcacattgcatggaaataaagatttttttggggggtggggtggggttgaaaatatgggtttcatatgatttcacacagatttcgaattctacacagctaagaggcaggattggaggaataagccagggtcttgcacatactaggcaaatgtatatttgaggaatattcccagctttctttaagcaatttttgagataaggtctcactatttgcgcaggttgatcttgaatttgagactctccaatgccagcctttgagcaactaggatgacatatgtgtgcccacttgactaattttgcaagcagttctttgatacacgtgtatgcctttagatattgtatgatggttgttctagttttcctttgttaagatattttaagtcaaagttgatactttgtagccatttaaattttttttctttctgtaactatctatgtaagcaaaacccagaattaattagttctgggagataatttgtgggattgtgtttcactttattattttttttacatataatgatacattttgtttaatggcttccaggagtctacttttcctgaggagaggtttagataccaggatgcattgtactatttgatttaaggacctgtatttccatgttaaatgttttcaaatggaagaacatgagttcgaaacttcttttcatatttgactctttagcattatgattattttcatgatttttgtgtttaag

SEQ ID NO: 8 - DNA, 16 bp, Artificial Sequence
  XTN recognition sequence 1
tggacgctaaaactca

SEQ ID NO: 9 - protein, 898 aa, Artificial Sequence
  XTN protein 1
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPEQVVAIASHDGGRPALESIVAQLSRPDPALAALTNDHLVALACLGGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 10 - DNA, 16 bp, Artificial Sequence
  XTN recognition site 2
tgactcttttttcctt

SEQ ID NO: 11 - protein, 898 aa, Artificial Sequence
  XTN protein 2
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNGGGKQALETVQRLLPVLCQAHGLTPEQVVAIASNGGGRPALESIVAQLSRPDPALAALTNDHLVALACLGGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 12 - DNA, 17 bp, Artificial Sequence
  XTN recognition site 3
tgaagtgtcctgcccaa

SEQ ID NO: 13 - protein, 932 aa, Artificial Sequence
  XTN protein 3
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNIGGKQALETVQRLLPVLCQAHGLTPEQVVAIASNIGGRPALESIVAQLSRPDPALAALTNDHLVALACLGGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 14 - DNA, 17 bp, Artificial Sequence
  XTN recognition site 4
tatatttttccagactt

SEQ ID NO: 15 - protein, 932 aa, Artificial Sequence
  XTN protein 4
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASNGGGKQALETVQRLLPVLCQAHGLTPEQVVAIASNGGGRPALESIVAQLSRPDPALAALTNDHLVALACLGGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 16 - DNA, 18 bp, Artificial Sequence
  XTN recognition site 5
tgcaaagaggttccgcta

SEQ ID NO: 17 - protein, 966 aa, Artificial Sequence
  XTN protein 5
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASNGGGKQALETVQRLLPVLCQDHGLTPEQVVAIASNGGGRPALESIVAQLSRPDPALAALTNDHLVALACLGGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 18 - DNA, 18 bp, Artificial Sequence
  XTN recognition site 6
tccatgtccatcaaagca

SEQ ID NO: 19 - protein, 966 aa, Artificial Sequence
  XTN protein 6
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASHDGGKQALETVQRLLPVLCQDHGLTPEQVVAIASHDGGRPALESIVAQLSRPDPALAALTNDHLVALACLGGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 20 - DNA, 22 bp, Artificial Sequence
  Minipig Rag1 recognition sequence for XTN
gtcggggactcaagaggaagag

SEQ ID NO: 21 - DNA, 7099 bp, Sus scrofa
  misc_feature (3597)..(3696): n = a, c, g, or t
gggggcagacaccagaagtaagagaggctacagctctattatccgtaaaaaggtcaccacgccaaaaacctataaaaatgaaaagacagaactataattcagatgagggagaaaggaaaaaccccagaaaatcagctaatcggtgaggaggttctcagcctccaggaaaaagactttagacctgatgctgaagatgatgcaggacattggaaataaactggaggcaaagatggataacttacagaaaacactgagcaaagagatacaagatataaaacttaaacaagaaggggtatgaaataccttgtttaaaaaaaaaaaagttaccttttgtggaagaaacaacatgaaatttggagacaaaaggcctgagctcaaattccagatcaggtatttactatccttgtaacttgggcaaatcattgaaccctccaccaaatgggaatcataataatggctaccccacagggtggtaatgagggaaaaacaaggaaaaatgaatgtggaaatattctaaaaatggaaggaaaaaaaaaaaaaacctctgcaaatgttaacttccactgtaagcagtttccatctaaacttagcaggtgagaaaagtacaaagatatcctgctttcaaaaataactgaatacactgaaagtaaaccgatcataatttaaaaaataaataacataatgcagaaagcaagccttcatgaaaaccagatttagcaaaactgacccaggattcatatggaaaaaacctggcagaaattttcagcaagacattaattctgcttttatacaatgagaaaacccagattttaaaagtctctgtggctttgagattgtagggggaaaacattggagaatctaaattaattaaaagtaaaaataaaaatcatccttggctactgatagctaaataaacacacactattgcaagatttaataactaactgcagctaacagttttcttcttccaacgttattattgaaaaacctagggagttcccattgtggcgaagtggaaaacgaatctgactaggaaccatgaggttttgggttcgatccctggcctcgctcagtgggttaaggatctagtgttgccatgagctgtggtataggtcgcagacacggctcggatctggcattgctgtggctctggtgtaggccggcagcaatagctccaaatgcatccctagcctgggaacctccatatgctgcgggtgcggacctaaaaagacaaaaaaccaaaaacaacaaaaaaagaaaaacctatggtagcagatgatcttctaaactcaaggttacaattctgacatcgtttaaactcagaaaatgacttattttccaaaaatattctacataagtgtaccaaatgggagcattttgaaaggtcattgtcgcatagtgtaagatattattagtagagaaatctgagaagtctttttctttttcttttgtggccactccatggcccatggcgttcccggaccagggatcagattcgagccacagttgtgaccttagctacagctgcagcaacgctggatccttaacccaccatgccaggctggggctggaacctgtgtcccagcactcccaagatgccacattgtgccacagcgggaattcttgaaaaccttttttaaatcttgtaaaaaaataagccaaaatatgtgaaatttataaaataaaaaattctgcaaaagtgatatgaaaaacacctctcagtataaactagaataacttgggcagggggaatggaaaccacaagtcaaattccttttctggcttcactggctgtcctttgacaaccttggctttgactttggcatcactcaatcctctccacatacatctttgttcacctctggtcccctgcagttacccctgttctcagctatggagctttcaaataactggtttctcctgttgcttttctgaactccctaaacagctcaagcaatcggcttaccagttacctattcctgacatgggttcaccatctctggcctcatcaacctaccaccatttgggtggtgaagccccacacccaggagttaagggtgccaggagaagctctccatgcaacccagagaaaacccatttgcaaaaccaatgcctaggcagcttccctgcttaaaattccatcaaatcttgagattctggagagttctctaggtctaatgtgtcccctgaacttctctgcaagtttatggtaaaccctgagtttttaaatgctttatgagcattcataaacttctgcaggtatttggaggtgtacaaccagtgatgtttcaaaacatcttccatttcatagtacttggactgcctggcattcatttttcggaagcgcctgaacagcttgttcccagactcatttccctcgctagcccatgccccaatggagccgtccctctcgataatttccgggacgtgggccagtgtcttgtgaaaataattggtgattttgccctcatatctgtacttgaacttggtggagaggagctcagcaaaacgctgcgaattgaaactatactggcagagggattccgggcactctttagcagggcatgacgatcgccagacgggtttcatcttcaggtaaaggtccatcagttccctcagagcttcatgcctctcctcggagggaattaactcacagactgcttccacagtctctttggtcatgagcttcctggcaaagttgccattcatcctcatgatgggcttcagattcatcttcttgcggaggtgcttgtccaaggtcgcctgccatctcttcctttcctccttggaggcatgggggttcttatacgcctcccctatctcgagctggaaaatcttgtagaactcggctgcattgccaatgtcacagtggagggcatctatggaaggcaccgtctcaatgaagggtttggccgagacccctttcacccggtcccgaagttcatccaccgtctcatggtatgggttggaacgccagacctcatagcgctccaaattctccgcgtggcttctggttatggagtggaagaccagattttgagaggcttccaggcgggtggcatcacagagagtacagatgtagacagagccagaagcctcaaggccttccacttcccggaccagtttctcatcatatccggtgcccctgaagatgaacttgaaagtccggaggatgcctcccatctccagcattagctggctgctcttcatggcctccctctcggcaatgagagggctcaggatggccgtcagggtctcatggtcggattcgtcggccagcatgaggcacaagggcttgcagcatagttcagagttaggcttggcttcctcaaacaccttcacgttctgtgacccgtgtgcgatggtgattttcatgactgtgaaggaaaaccgaacggccttttccggcacgaccggcccactgccgtgcttctcactcacgtctcccatcccatcacaagactccttcaccaccacagtgaaggggccattcaggtagtcgtcaaggtcttgggctctcataccctccaggatgtcttctcacatgtccatgagagcggacaccagagccgagtcatagcggaagcgctggcaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtcttcttgtgggaggccttgacttgcagcttgagctcactcgacggtgtttctgagccctccgcgtcagggacaggagatgctggcggggccggccccnctttattaatatgcacaaatgtctccttcgactccttgtggcttgagatatggtgattatatttttccaagctgatctcctcgttgcactcctttgctgggcatttcaccatcagggtattcaagatgctcagaaaagacttcactggactctccaggtcagtagggaaacagggatagtggcaagagggacaactgctgcccatgactttgaggcacctgagaatgcagatcctgcaaaacacgtgcttgcagctggtctccaccgggtcggccaggatgtgttcacaaatctggcaggagatagatttcacaaagtgcgccgggaagtccactgccaggagcttggggctaagatgtatctgaccgcagttggcgatcttcttcatcagttccttgctgctgatcctggcctgagctctcctcttgcgctgacgggcttgtctcgctcggtcaatcacagttttgagttttttgctgagctgcatgtttggctgctgactcttcctcttgagtccccgacgtgcaatgtggcagatgtcacagtttagggtgtgggggtgccactccatggttgcattccttggggagtaaacctcacatggggtgctgctaaacttcctgtgcatgaagctccagcagttatggcagaactcagtggggtggatcgagtcaacatctgccttcacatcgatccggaaaactttggcaatgaggtctggccaggacgtggccctcttttccttcttccgtaaaaggacttgggttttaccatccacaggcccgtggactggataccttctcttgtgcccagtggtgttgaaagaattcccacagatgcggcagagacgtctcaggttggcttggtggatggctttgtctcttgctttcccatcttcgtgggattcctttaaaaactttggatggggcttgaatgctggttgaggcagggctgacttctgaccaccaggcttgtccaggactgctggagattgctccagcgagggtttcccctcggaggaatcctgcttttccgtttgagccttttcaggtgccttttcaaaggatctcaccctgaatagcttaaacttccattctgaaaatttaatgtgggggtgctggatttcatctggggcggaactgagtcccagagtgggtggcaaagagacagccatgctggctgaggtacctgggagcaataaggacaagtcaggttaggaaaaaaaatgtaaataacccactgatgcaacttggaaggaataccaattagtacattcaattattcatttattttttattttattttattatttattttatttttttgtcttttttgcctttttccagaaccagcacacagaggttcccaggctaggggagctgcagccgccggcctacgccagagccacaaccgcttgggacctgagccgcatctgcgacctacaccacagatcatggcaataccggatccttaacccactgagtaaaggcagggatcgaacccacaacctcatggttcctagttggattcattaaccactgcgccacaatgggaacgcctattcatttattaaagcattcagaaagttaattgtttggcttcctatgttaatatactatgttagggctgatcatatacagaaaacagagtcatttcctgttctttcagtggcattgtccatttacatgtttctgcgttcacgcatcatcaccatggtctattttacatatatgtgtccaatcatttcttttataaaaattattcttttttattgagatatagttgatgtacaatattatctatgtacgatattatttcctttccaccaaaacctggacattttttaggacggaatctttgagtgatttatctctgtctcccccaagctttctagcacacattagatgctcaatagttatctgctgaataaatgcaaggaaaaactggttactaaaaccaaagaaaagtcatattttccgcgctgtatcagtctcattgcactagctggacaatgtatttccagctttttaaagcagtctgccaggaggttggttgtttatttctctgaaacgggacataaagggatgataaagctacaaatccaaagaaactaattactcgctacttgaaaggatgaagtgaattttgagctttatgaatgcacactaataacaagtatcatggatagtaattatgcttacaattttaattagtgaacgctgctgtaaattataaggctgtgtctccattaggaacaaaacccacagaactctctaaacctgcaacaaaccacagtgtgcatgtacagagactccaaaggacttttaagatgaaaaattcaagtagtttcaggtcgaattcacacaggccatcccaaaagtcattactgggcactggtcattagctctatcgtttgtaataagaagaggtgggagtaataattttcttcttcaaaaaaaacattggccattatatattccaggaaaccctagcaacctgggttgtggtgtaggcttcaacagagctgtagaaaaaagtttaaaaatgccatttgagtgtctctagacccaattagcacaatattctcaaaattccttagtatcagcatgactggaaaaacagcaattgtattttcaaactacaatctaaattttccatattttcctgggttagtgctcttgacccgccaattatatcaaacaatgcttccccttcaattaactaactaaccaagttgatgatattgttattgtcctgtggtatggagactaatcctctgatgaacacactttgcccaggggtgggatttgggggaggggaaaggaaagccacaagggtgatcctgactgctgctaacaaagcttcctgttaggtcatccctttcaaccacagaagtttgttccatcttgcatgcatgatgcttcagagattcatgtttttcataaagacgaaaagccaatttccaagtcttggttgaaaaggaaatcctgttacactgttctatatgcaggactgtgtgagaactaaacagttccaaaccatttcaaaggcctcattaacctcaaaagtatttcaactgcagtcagaccaacgcctcaaagttgcttatagttcagttatgttcctacattcaggatcagcggcaaggatgtcacttctgtcccactggtttgaacttatttccatgtgttttgtttctttagcccttcctttccctgtgtctgggccaaggattgaaccgacatcacagcagtgacctgagccacaacagagacaatgctgggtccttaacccattgagcta

SEQ ID NO: 22 - DNA, 300 bp, Artificial Sequence
  Partial Rat Nrf2 gene
ccgcctttaagttcttgtccggtccctaggtccttgttccgccctcagggggcgggggcggggcggactaagactggcctgccactccagcgagcaggctatctgctggttccccactgctccgactagccattgacgccgcctcacctctgctgccagtagcctcgccgtcggggagccctagcgcagcgtccgccctcagcatgatggacttggaattgccaccgccaggactacagtcccagcaggtgctgccctggccttcgggcccctcagctatgccaagaatggaagggcgaa

SEQ ID NO: 23 - DNA, 270 bp, Artificial Sequence
  D07 Nrf2 clone
ccgcctttaagttcttgtccggtccctaggtccttgttccgccctcagggggcgggggcggggcggactaagactggcctgccactccagcgagcaggctatctgctggttccccactgctccgactagccattgacgccgcctcacctctgctgccagtagcctcgccgtcggggagccctagcgcagcgtccgccctcagcatgatgacccagcaggtgctgccctggccttcgggcccctcagctatgccaagaatggaagggcgaa

SEQ ID NO: 24 - DNA, 269 bp, Artificial Sequence
  C01 Nrf2 clone
ccgcctttaagttcttgtccggtccctaggtccttgttccgccctcagggggcgggggcggggcggactaagactggcctgccactccagcgagcaggctatctgctggttccccactgctccgactagccattgacgccgcctcacctctgctgccagtagcctcgccgtcggggagccctagcgcagcgtccgcccaggactacagtcccagcaggtgctgccctggccttcgggcccctcagctatgccaagaatggaagggcgaa

SEQ ID NO: 25 - DNA, 54 bp, Artificial Sequence
  D12 Nrf2 clone
ccgcctttaagttcttgtcctccgcctcagctatgccaagaatggaagggcgaa

SEQ ID NO: 26 - DNA, 121 bp, Artificial Sequence
  D04 Nrf2 clone
cattcttggcatagctgaggggcccgaaggccagggcagcacctgctgggactgtagtcctggcggtggcaattccaaggacctagggaccggacaagaacttaaaggcggaaagggcgaa

SEQ ID NO: 27 - DNA, 55 bp, Artificial Sequence
  Partial Rag1 rat Gene with endonuclease targeting site
gctcaaccatgctagacgggaccgtcgcaagagaactcaggctagggtcagcagc

SEQ ID NO: 28 - DNA, 39 bp, Artificial Sequence
  1A4 clone of Rat rag1 sequence
gctcaaccatgcaagagaactcaggctagggtcagcagc

SEQ ID NO: 29 - DNA, 39 bp, Artificial Sequence; identical to SEQ ID NO: 28
  1A11 clone of rag1 sequence

SEQ ID NO: 30 - DNA, 42 bp, Artificial Sequence
  1B8 clone of rag1 sequence
gctcaaccatgctagacgggaactcaggctagggtcagcagc

SEQ ID NO: 31 - DNA, 42 bp, Artificial Sequence
  1C8 clone of rag1 sequence
gctcaaccatgctagacgggaactcaggctagggccagcagc

SEQ ID NO: 32 - DNA, 39 bp, Artificial Sequence; identical to SEQ ID NO: 28
  1C9 clone of rag1 sequence

SEQ ID NO: 33 - DNA, 15 bp, Artificial Sequence
  1G9 clone of rag1 gene
gctcaaccatgcagc

SEQ ID NO: 34 - DNA, 25 bp, Artificial Sequence
  2E1 clone of rag1 sequence
gctcaaccatgctagggtcagcagc

SEQ ID NO: 35 - DNA, 39 bp, Artificial Sequence; identical to SEQ ID NO: 28
  2F6 clone of rag1 sequence